(19) United States
(12) Patent Application Publication
(10) Pub. No.: US 2005/0085624 A1
(43) Pub. Date: Apr. 21, 2005

(54) Method and device

(76) Inventor: Darcy Birse, Quebec (CA)

Correspondence Address: AMERSHAM BIOSCIENCES;PATENT DEPARTMENT, 800 CENTENNIAL AVENUE, PISCATAWAY, NJ, 08855, US

(21) Appl. No.: 10/498,833

(22) PCT Filed: Dec. 12, 2002
(86) PCT No.: PCT/IB02/05740

(30) Foreign Application Priority Data
Dec. 12, 2001 (SE) 01041862

(51) Int. Cl. C12P 21/06; C07K 1/02

(52) U.S. Cl. 530/333; 435/68.1

(57) ABSTRACT

A method of modification of a protein or polypeptide in the presence of a modifying composition capable of providing at least one modification wherein a liquid phase comprising the protein or polypeptide is brought into contact with a solid phase capable of immobilizing the protein or polypeptide and the solid phase carrying the immobilized protein is brought at least once into contact with a liquid phase comprising the composition capable of modifying the protein or polypeptide and modification reaction(s) are allowed to occur. The liquid phase comprising the protein or polypeptide may be a liquid extract of eukaryote or prokaryote cells. The modification may be a acylation, phosphorylation, dephosphorylation, SUMOylation, ubiquitinylation, carboxymethylation, formylation, acetylation, deacetylation, gamma carboxyglutamic acid, norleucine, amidation, deamidation, carboxylation, carboxyamylation, sulfation, methylation, demethylation, hydroxylation, ADP-ribosylation, maturation, adenylation, O-linked glycosylation, N-linked glycosylation, methonine oxidation, and addition of lipid (prenylation).

### TECHNICAL FIELD OF THE INVENTION

The present invention relates to a method of chemical or enzymatic modification of proteins and polypeptides and to a kit for use in such a method. In particular it relates to a method of purification and post-translational modification (PTM) of proteins and polypeptides and to a kit for use in this method.

### BACKGROUND OF THE INVENTION

Within the field of life science, there is an increasing recognition of the importance of post-translational modifications of proteins in eukaryotic cells. Many modified proteins are critical in cell signaling processes or crucial to biological processes and are often modified in highly specific sub-cellular localizations or as a function of temporal gradient or timing event. However, modified proteins are very hard to identify and isolate from endogenous material. This difficult isolation of a homogeneously modified target protein increases the difficulty to purify this form of protein and dramatically decreases the yield.

In view of this, a major problem with conventional cellular characterization studies is the definition or isolation of underrepresented or low-abundance proteins and their differential expression patterns, often comprising post-translational protein modifications. It is these difficult to characterize proteins that are often the most interesting to understand and confer a functional characteristic of a specific role in the cell. The production of these modified proteins can often be seen as being critical to understanding their function, or dysfunction, in a biological system.

Also, in some functional studies and/or diagnostic assays, it is becoming apparent through experimental observations that modified proteins can be considered essential. In terms of recombinant material, often these proteins are produced without modification and function poorly compared to their modified form, whereas, as stated herein above, the isolation of endogenous modified forms of the protein can be extremely difficult and yield low levels in terms of quantity and poor levels in terms of purity and homogeneity.

For functional studies and/or diagnostic assays, post-translationally modified proteins provide a tuning that cells need to function. Producing modified proteins are critical to understanding complex protein mediated pathways. There are often not enzymatic intermediates to follow signaling pathways and events, but protein-protein interactions mediating or modulating a certain effect in the cell. It is in these cases that post-translational modifications are becoming evident to be playing increasingly complex roles in controlling and signaling biological systems.

A severe problem in the field of structural studies of modified proteins is due to the amount of material needed and the level of purity needed to conduct an experiment. Large quantities of highly purified, homogenous material are necessary to conduct a structural study. The production of suitable material is often the rate-limiting step. The production of eukaryotic modified material is expensive, inefficient and often results in a highly heterogeneous product.

Within the field of drug screening and production, the use of recombinant material is an attractive alternative considering the cost and requirement of large quantities of material needed to study. A drawback is the form of material produced, which generally fails to provide the eukaryotic like modifications that can best mimic the system that the drugs are being designed and intended in use for, often mammalian systems. This is a problem especially in view of the fact that typical drug-screening campaigns are expensive and that the success of the assay often depends on the quality of the target protein being used. In addition, this applies to diagnostic assays and screens that may be used as analytical tools or in high-throughput screening applications.

Also in the actual production of recombinant peptides for the use as drugs, post-translational modifications (such as amidation, necessary for amino and/or carboxy-terminal protection) are often required in the formulation of that product.

Marcucci et al., in U.S. Pat. No. 6,172,202 disclose a process for the preparation of a conjugate between a poly (ethylene glycol) (PEG) and a protein or glycoprotein comprising specifically binding the domain of the protein or glycoprotein to a specific binder to shield it from the PEG in the conjugating step, and wherein the specific binder is released subsequent to the conjugation. The advantage achieved by the process of Marcucci et al is said to be a more homogeneous product and preserving of biological activity. No mention is made of immobilization of a protein or of enzymatic modification of a protein.

Dalborg et al., in U.S. Pat. No. 6,048,720 disclose a process for improving the in vivo function of factor VII by shielding exposed targets of the same, comprising immobilizing factor VII by interaction with a group-specific adsorbent and subsequently conjugating an activated biocompatible polymer, preferably a PEG homopolymer, to the exposed targets of the immobilized factor VII. The purpose of the immobilization is to exclude the interaction sites on the polypeptide from conjugation to the biocompatible polymer.

It seems that Dalborg et al. and Marcucci et al. generally address the same problem of conjugating a polymer, basically a PEG polymer, to a polypeptide whilst shielding the active site of the latter from the conjugation reaction.

Colpan et al., in DE 3717210A1, disclose a method of modifying a biopolymer by immobilizing it on an adsorbent, reacting the immobilized biopolymer, optionally use it further and desorbing the reaction product. It appears that the biopolymer to be modified by method of Colpan et al. is a nucleic acid. The immobilization of nucleic acids and modification of nucleic acids is quite different from protein/peptide isolations and modifications. Nucleic acids are very robust molecules (biopolymers) that can be subjected to a wide range of experimental conditions, whereas proteins/peptides are vulnerable and sensitive under a similar range of conditions. The nucleic acid method described by Colpan et al. involves completely denatured nucleic acid material incompatible with chemically and biologically relevant conditions.

T. P. Bradshaw and R. B. Dunlap in Biochemistry, 32 (1993) 12774-12781 investigate a heterodimer of thymidylate synthase using chemical modification of the enzyme immobilized on a Sepharose resin. Their method however is very inefficient, requiring several purification steps and providing only a low yield. The aim of the method is apparently only for analytical biochemical analysis and no suggestion is made of any advantageous modified protein production.

WO 00/50902 discloses a method for analysing a sample containing “biologically significant molecules”, such as enzymes and enzyme modulators, which generally consists of providing a pair of proteins or polypeptides capable of associating with one another in a way dependent on the state of modification of at least one of the two entities in the pair, one of them being immobilized on a solid phase. The modification, under the action of the “biologically significant molecules” in the sample, of any the two proteins/polypeptides is susceptible of influencing the association of said proteins/polypeptides. The state of association therefore is used a means for assessing the modification which has taken place. In said method, use is made of a pure and well-known protein composition as a means for studying e.g. enzymes and enzyme modulators and samples susceptible to contain them.

The methods of the prior art as summarized herein above, when directed to the production of proteins, all teach performing an important number of purification steps on the protein and suffer from low yield.

### SUMMARY OF THE INVENTION

In a first aspect of the present invention there is provided a method of chemical or enzymatic modification of a protein or polypeptide as specified in claim 1.

According to a still further aspect of the invention, kits are provided for use in any of the inventive methods and/or applications.

Further aspects of the invention are as defined in the claims.

### BRIEF DESCRIPTION OF THE DRAWINGS

To facilitate understanding of the invention, exemplary embodiments will be described further below, with reference to the drawings, wherein:

FIG. 1 represents immobilization of a protein or polypeptide on a solid phase.

FIG. 2 represents modification in-line on-column of protein or polypeptide in the presence of modifying composition.

FIG. 3 represents modification in-line on-column-reiterated steps.

FIG. 4 represents cleavage of suitably modified protein or polypeptide from fusion linker.

FIG. 5 represents elution of modified protein or polypeptide.

FIG. 6 represents purification of rat HNF4α LBD::GST fusion protein on a GSTPrep 16/10 column.

FIG. 7 represents purification of rat HNF4α LBD on a Resource Q 6 ml column.

FIG. 8 represents purification of rat HNF4α LBD on a HiLoad 16/60 Superdex 75 prep grade column.

FIG. 9 represents SDS-PAGE analysis of the protein samples from rat HNF4α LBD purification.

FIG. 10A represents MALDI-TOF MS identification of the purified rat HNF4α LBD-m/z spectrum.

FIG. 10B represents the full length amino acid sequence of the rat HNF4α protein.

FIG. 11 represents purification of mouse TDG on HiPrep Heparin 16/10 column.

FIG. 12 represents purification of mouse GST::TDG fusion protein on a GSTPrep 16/10 column.

FIG. 13 represents purification of mouse TDG on HiLoad 16/60 Superdex 200 prep grade column.

FIG. 14 represents SDS-PAGE analysis of the protein samples from mouse TDG purification.

### DETAILED DESCRIPTION OF THE INVENTION

In one important aspect the invention aims at providing a method allowing for the purification, post-translational modification and isolation of a modified protein or polypeptide in a systematic, reproducible and effective manner.

Furthermore, in another aspect, the present invention aims at providing a method allowing for the scale-up of post-translationally modified protein or polypeptide production to obtain large amounts of modified material that has the distinct advantage of higher yields and higher levels of purity which, by following this method, can be used for production, analytical and large-scale industrial applications.

Moreover, the present invention aims at providing a method allowing for an extensively automated production of post-translationally modified proteins or polypeptides.

Generally, a method of the invention will comprise the steps of
i) bringing a liquid phase comprising the protein or polypeptide into contact with a solid phase upon which the protein or polypeptide is susceptible of being immobilized and allowing immobilization of the protein or polypeptide on the solid phase to occur; and
ii) bringing the solid phase carrying the immobilized protein or polypeptide into contact with solution comprising at least one modifying enzyme, susceptible of catalyzing a modification reaction of the protein or polypeptide, and any other components necessary for the modification reaction(s) and allowing the modification reaction(s) to occur.

The source of the protein or polypeptide is not critical to the invention. For example, using conventional genetic engineering techniques, the protein or polypeptide may be produced by a host organism, e.g. a yeast cell or a bacterium such as Escheerichia coli or Bacillus subtilis, which has been transformed or transfected with an expression vector, obtained by insertion of a coding gene or part thereof into a vector in a conventional manner. The transformed or transfected host is cultured and proliferated under suitable conditions, as known to the person skilled in the art. However, it should be understood that the invention is by no means limited to recombinantly produced proteins or polypeptides, but could be applied in principle to any protein obtained from any source.

The way of obtaining a liquid phase comprising the protein or polypeptide also is not critical to the invention. For example, the protein or polypeptide may be present in a liquid phase containing a cellular extract from a eukaryote or prokaryote cell, e.g. a bacterial host over-expressing a recombinant protein or polypeptide. The cellular extract may be obtained by methods well known to the person skilled in the art, e.g. by cell lysis, centrifugation, ultracentrifugation, collecting of supernatant fractions etc. It is a very advantageous feature of the invention that the method may be applied directly to a cellular extract having been subjected to no purification step. Indeed, in most of the methods of the prior art, purification of cellular extract are required leading to substantial losses and decrease of overall yield. In contrast, in the method of the invention the protein in the cellular extract may be selectively immobilized on the solid phase, the other, contaminating components washed away, and the modification may be performed.

The liquid phase comprising the protein or polypeptide may be a suitable aqueous buffer solution, such as PBS (Phosphate Buffered Saline), HEPES (4-(2-Hydroxyethyl)-1-piperazineethanesulfonic Acid), Tris (Tris[hydroxym ethyl]aminomethane) and MES (2-[N-Morpholino]ethanesulphonic acid).

This liquid phase containing the protein or polypeptide to be post-translationally modified is brought into contact with a solid phase, upon which the protein or polypeptide selectively is retained. The solid phase may be any suitable matrix, e.g. a resin, such as an affinity resin or ion exchange resin, upon which the protein or polypeptide is immobilized by a characteristic of its nature or by being fused to a fusion linker/moiety.

In a preferred embodiment, at least one exchange of liquid phase in contact with the solid phase carrying the immobilized protein or polypeptide is then performed: this step may comprise bringing the solid phase carrying the immobilized protein or polypeptide in contact with a liquid phase, of the same basic composition as the original liquid phase comprising the target protein or polypeptide, or of a different composition. Exchanging the liquid phase advantageously improves the enrichment level and homogeneity of the immobilized protein or polypeptide, removing further contaminants. In addition, with the foresight of knowing what the downstream applications are, the liquid phase can be exchanged to be compatible with downstream processes (e.g. remove phosphate component in a binding buffer to exchange with Tris-based buffer, remove additives, detergent exchange, introduce additives essential for downstream process such as reducing agents, salts, etc.). This step has the advantage of also completing an additional washing step enriching the immobilized target protein or polypeptide further. This step can be reiterated depending on the downstream process (multiple phosphorylation steps with different kinases under different conditions, multiple modifications, on-column cleavage reactions, etc.).

In the subsequent step ii), the solid phase carrying the immobilized protein or polypeptide is brought into contact with a liquid phase comprising at least one modifying enzyme and any other components necessary for the modification reaction(s) to occur, and then allowing the modification reaction(s) to occur.

Exemplary for components which are necessary for the modification reaction(s) to occur are reducing agents, co-factors, substrates etc., such as ATP (adenosine 5′-triphosphate; nucleotide donor), EDTA (Ethylene Diamine Tetraacetic Acid; metal ion chelator), phosphatidyl serine (activating co-factor) and detergents (Triton-X-100, NP40, dodecylmaltoside; which increase protein or polypeptide stability and solubility).

The liquid phase per se may be of the same kind as the precedent liquid phase(s) or may differ therefrom. It preferably comprises buffer solution plus additional components that increase the efficiency of the modification reaction.

The modification reaction may be performed in batch or in-line on-column, and can be stringently controlled. To ensure reproducibility, the incubation time and temperature may be monitored, and all the reaction components to be supplemented systematically introduced. The process may be automated.

In one advantageous embodiment, the reaction, also termed incubation, is performed in-line on-column. This allows for time-dependent incubation for post-translational modifications to the immobilized target protein or polypeptide. In addition, for fusion proteins or polypeptides that require proteolytic cleavage to remove the fusion tag (either N- or C-terminal fusions with a (proteolytic) cleavage site), this step can be used to process the immobilized fusion protein or polypeptide by removing the affinity tag.

In one embodiment, the liquid phase comprising enzyme is circulated, i.e. the solid phase solid phase carrying the immobilized protein or polypeptide is brought into contact therewith more than once, e.g. from 2 times, or by use of a recirculation-loop providing constant modifying interactions for a given time frame and flow rate variables. This allows for a shorter total incubation time since the diffusion of enzyme and other components within the liquid phase to the immobilized protein or polypeptide becomes less important as a rate limiting parameter. The number of circulations may be selected so as to obtain a suitable yield of the modification process.

Next, the modifying enzyme(s) and auxiliary components may be washed away, by bringing the solid phase carrying the modified protein or polypeptide in contact with a suitable liquid phase. This step can work as a buffer exchange, but can also be used as a further purification enrichment step to increase the purity of the immobilized target protein or polypeptide. All unwanted reaction components may be washed away whereas the immobilized modified protein or polypeptide remains bound to the resin.

In one embodiment, a liquid phase comprising modifying enzyme(s) and auxiliary components of a different kind is subsequently brought into contact with the solid phase carrying the still immobilized protein or polypeptide, which will comprise modified as well as non-modified protein or polypeptide, and the protein or polypeptide is further modified.

This sequence of successive modifications can be performed in principle as many times as desired and may be optimized to obtain a given desired yield of a protein or polypeptide suitably modified.

Subsequently, the modified protein or polypeptide may be collected from the solid phase by an elution step using a suitably selected liquid phase, such as a suitable buffer solution. Advantageously, the liquid elution phase may be selected in view of the downstream events, such as the projected use of the modified protein or polypeptide. Subsequent to elution, modified protein(s) or polypeptide(s) may be separated from non-modified proteins or polypeptide(s) by any conventional protein or polypeptide separation method, known to the person skilled in the art, such as analytical gel filtration, or other chromatographic methods separating modified vs. unmodified proteins or polypeptides. These auxiliary steps can be used to approach the highest level of modified protein enrichment (highly pure modified target protein). In principle, the homogeneity of the modification reaction is completed to its highest level prior to elution from the immobilized status.

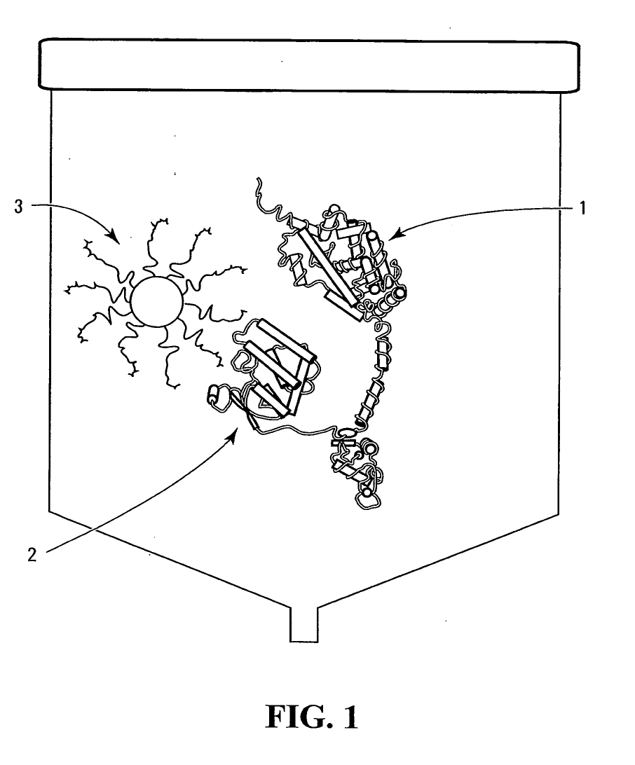

Some exemplary embodiments of the invention will now be briefly outlined, referring to the figures. In FIG. 1 a protein or polypeptide 1, fused to a fusion linker 2, is immobilized on a resin by affinity of fusion linker 2 for ligand group 3 on the resin. In exemplary alternative embodiments, the protein or polypeptide 2 may be immobilized by a characteristic of its nature (ex. immuno-affinity, functional domain (nucleotide or heparin binding), or chemical nature (ion exchange)). The immobilized protein or polypeptide 2 can then be enriched further removing contaminants by use of binding buffer.

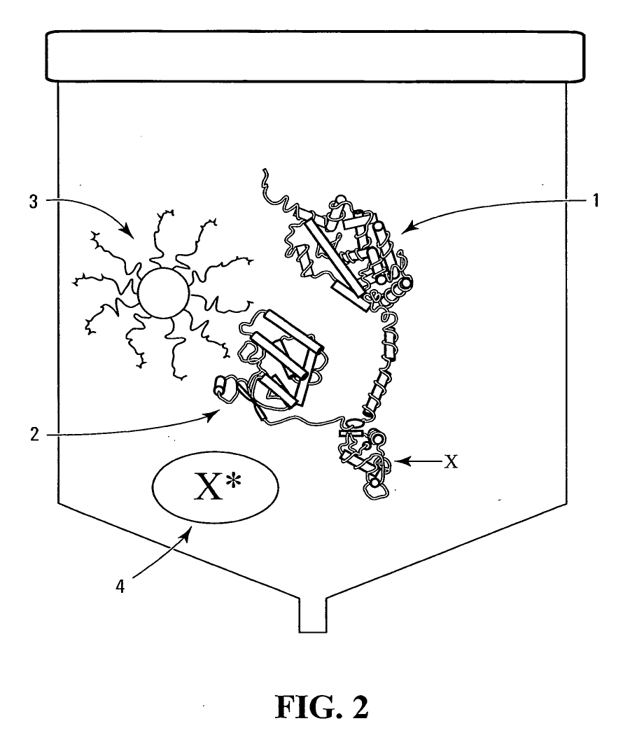

Buffer exchange preferably is then performed to improve the enrichment level and homogeneity of the immobilized protein or polypeptide 2 by removing contaminants. Optionally, the buffer is exchanged to be compatible with downstream processes This step may be reiterated, such as for multiple modification steps with different modifying components under different conditions, on-column cleavage reactions, etc. Reaction buffer and modifying component(s) 4 are added (FIG. 2), to provide for modification X of the protein. The incubation is performed in batch or in-line on-column, whereby the X modification reaction may be stringently controlled by appropriate monitoring of the incubation time, temperature, and all the reaction components to be supplemented.

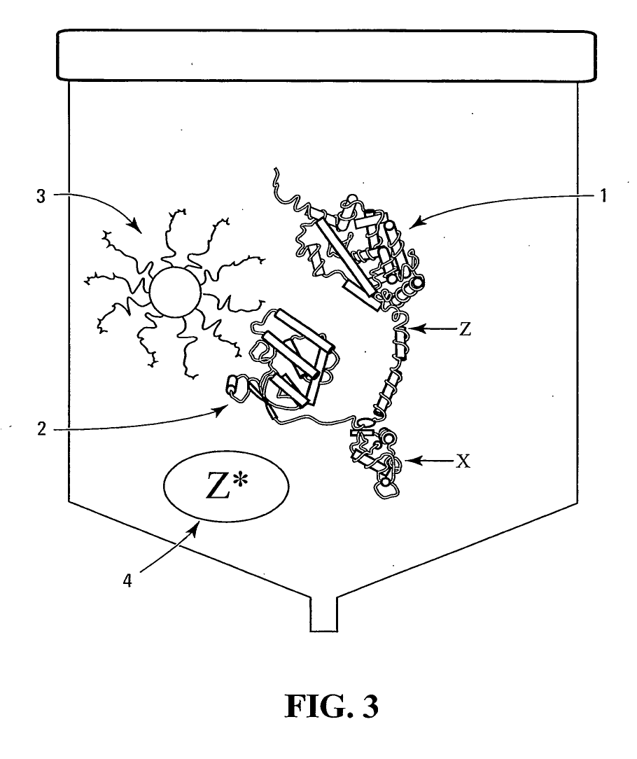

The immobilized X-modified protein or polypeptide 2 may be submitted to Z-modification using modifying component 4′ (FIG. 3) optionally subsequent to buffer exchange and/or purification enrichment increasing the purity of the immobilized X-modified protein 1 or polypeptide.

The preceding steps can be reiterated in a cyclic manner for a number of modifications to a target protein or polypeptide, whereby all unwanted reaction components are washed away and the immobilized modified target remains bound to the resin.

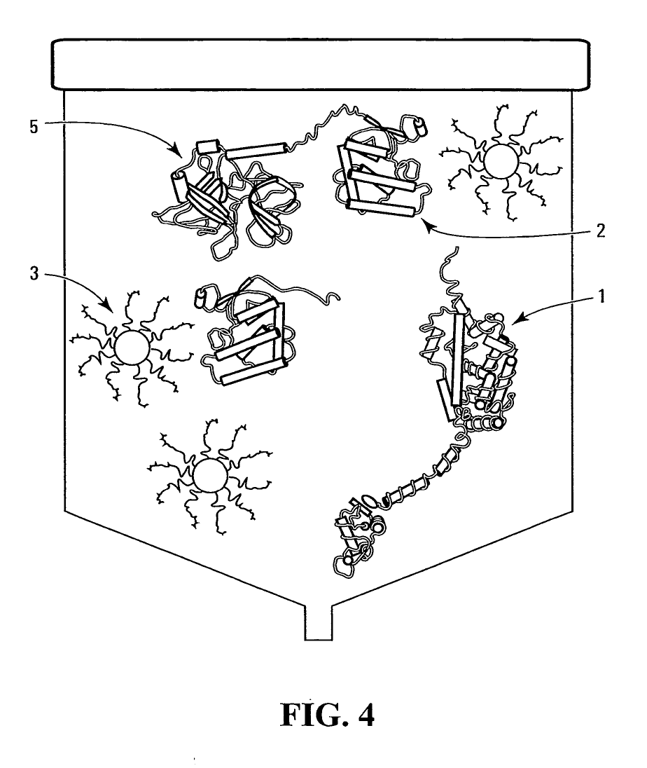

To collect the suitably X,Y-modified protein or polypeptide 1 without the fusion linker 2, a cleavage component 5 for breaking the bond between these is added, such as a protease (FIG. 4).

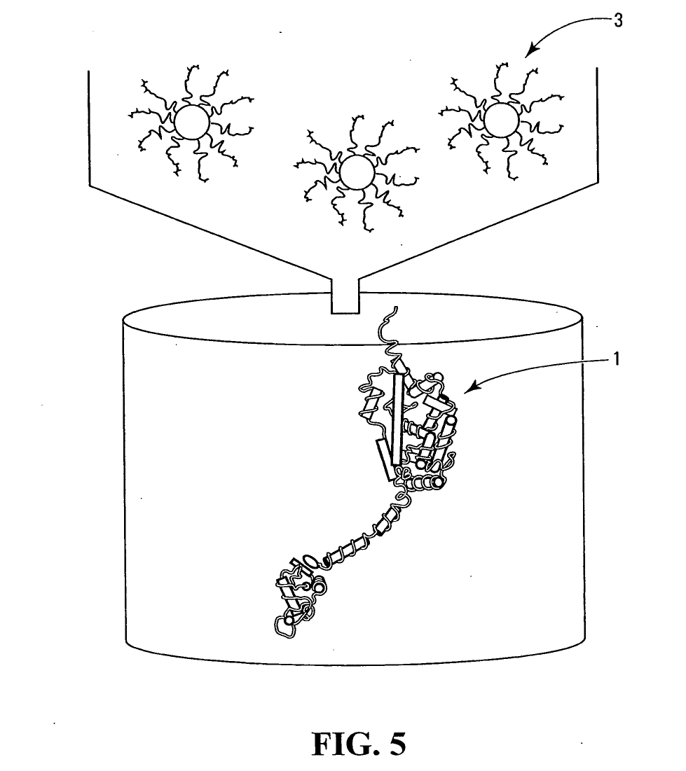

Finally, the X,Y-modified and cleaved protein or polypeptide is collected by elution from the resin (FIG. 5).

The yield of modified protein or polypeptide can be assayed by several analytical methods, known to the person skilled in the art, such as electrophoresis (native and denaturing), immunoblotting, mass spectrometry, functional/activity assays, NMR, and crystallography. The final product is highly enriched, highly pure, post-translationally modified, in high yield and concentration.

When the protein or polypeptide is fused to a fusion linker, i.e. a protein or peptide fragment having affinity for the solid phase, the elution step comprises bringing the solid phase carrying the modified fused protein or polypeptide into contact with a liquid phase comprising an enzyme susceptible of cleaving the bond between the modified protein or polypeptide and the protein or peptide fragment having affinity for the solid phase as well as any other components necessary for the cleaving reaction to occur.

In principle, any type of protein or polypeptide modifications, enzyme-catalyzed or not, may be performed by the method of the invention, such as e.g. a acylation, phosphorylation, dephosphorylation, SUMOylation, ubiquitinylation, carboxymethylation, formylation, acetylation, deacetylation, gamma carboxyglutamic acid, norleucine, amidation, deamidation, carboxylation, carboxyamylation, sulfation, methylation, demethylation, hydroxylation, ADP-ribosylation, maturation, adenylation, O-linked glycosylation, N-linked glycosylation, methonine oxidation, and addition of lipid (prenylation).

To illustrate the importance of post-translational modification reactions in eukaryote organisms, some of them will be discussed herein below. Phosphorylation Among all post-translational modifications in the eukaryote cell, phosphorylation is a frequently used and powerful mechanism for the rapid modulation of transcription factors activity in response to environmental conditions and hormonal signals. Most, if not all, nuclear receptors studied to date are phosphoproteins whose functions are regulated by phosphorylation. In view of this, a method allowing to produce precisely and reproducibly post-translationally phosphorylated molecules will be of great advantage e.g. for the screening of possible drugs, for the study of regulatory phenomena of the eukaryotic cell etc.

SUMOylation (Small Ubiquitin-Related Modifier)

A number of eukaryotic proteins are post-translationally modified by the ubiquitin-like modifier SUMO-1 protein (small ubiquitin-related modifier) and its close homologues SUMO-⅔. The pathway of SUMOylation is mechanistically analogous to ubiquitinylation, however requiring a distinct set of enzymes. Presently, the dimeric SUMO-activating enzyme (UBA2/AOS 1) and the SUMO-specific E2 enzyme UBC9 have been characterized.

The SUMO-1 protein functions in protein-protein interactions, signal transduction, can act as an antagonist to ubiquitinylation and protein degradation, increasing proteins stability and half-life.

Ubiquitinylation

In the process of ubiquitinylation a protein is modified by covalent bonding to ubiquitin through the ligation of the C-terminus of ubiquitin to the á-amino groups of protein lysine residues. Ubiquitin is a small, 76-residue protein common to all eukaryote organisms. The major, but not sole, function of ubiquitinylation is to target proteins for degradation, usually by formation of a multiubiquitin chain on the target protein. Recently, dysfunctional ubiquitin pathway of oncoproteins has been recognized to be implicated in development of tumors.

Ubiquitinylation of specific lysine residues involves a multi-enzyme system, but the key component in regulation and biological specificity is the ubiquitin-protein ligase (also known as E3).

Carboxymethylation/N-Ethylmaleimide (NEM)

This is a modification providing protection of solvent exposed cysteine residues to prevent aggregation and oxidation of proteins. It is advantageously used for the production of hydrophobic proteins that are subject to oxidation-dependent aggregation and therefore, precipitation. It allows for the production and isolation, in a concentrated form, of hydrophobic proteins that are known to aggregate and precipitate.

Acetylation

Acetylation is used to modify positively charged Lys/Arg amino acid residues. It has an important function in chromosomal DNA remodeling and transcriptional regulation. It is a reversible modification.

Amidation

This provides a means of amino and/or carboxy-terminal protection of polypeptides. This is valuable in drug production/bioprocessing applications where the amino and/or carboxy-terminal must be protected for transport as a drug increasing stability. Many bioactive peptides must be amidated at their carboxy terminus to exhibit full activity. The peptides are synthesized from glycine-extended intermediates that are transformed into active amidated hormones by oxidative cleavage of the glycine N—C alpha bond. In higher organisms, this reaction is catalyzed by a single bifunctional enzyme, peptidylglycine alpha-amidating monooxygenase (PAM). The PAM gene encodes for two enzymes that catalyze the amidation reaction. Peptidylglycine alpha-hydroxylating monooxygenase catalyzes the stereospecific hydroxylation of the glycine alpha-carbon of all the peptidylglycine substrates. Peptidyl-alpha-hydroxyglycine alpha-amidating lyase generates alpha-amidated peptide product and glyoxylate.

Methylation

Methylation is used to modify positively charged Lys/Arg amino acid residues. It has an important function in chromosomal DNA remodeling and transcriptional regulation and is a reversible modification.

Glycosylation

Glycosylation is used to modify proteins by bonding them to carbohydrates (oligosaccharides). There are two main types; O-linlced (to the OH side chain of Ser and Thr) and N-linked (to the NH2 side chain of Asn in the sequence Asn-X-Ser/Thr, where X can be any amino acid besides Pro and Asp). Some proteins are attached to the plasma membrane by a third type of carbohydrate structure called a glycosyl phosphatidylinositol (GPI) anchor. Modification in vivo can only be performed in the lumen of the rough endoplasmic reticulum, and can subsequently be modified in the lumen of the golgi apparatus where other amino acids of the protein may become glycosylated. Glycosylation is a reversible modification.

The above enumerated and briefly outlined post-translational modifications are exemplary only, and the invention should not be construed as limited to these. More details about post-translational modifications of proteins may be found in T. E. Creighton, Proteins: Structures and Molecular Properties. W. H. Freeman and Company, New York, second edition. (1993) ISBN 0-7167-7030-X.

The method of the invention has several advantageous features: Firstly, it may be performed in one single chromatographic step. The method can be performed in batch mode, in a self-packed column or in a pre-packed column format.

The batch mode format can be applied in multi-well titre plates for high throughput applications. It allows the users to use the resin of their choice, and it does not necessarily need the high-pressure characteristics of controlled flow systems (ex. chromatographic platforms). Also, this can be applied directly on the lab bench without the requisite of having a chromatographic platform. It facilitates the throughput (doing multiple parallel experiments), and can allow for simple experimental design and side-by-side comparisons for different conditions. A method for screening for the immobilization, modification and purification process of a, or series of, target(s) (and can also be used in immobilized/modified form for drug discovery approaches directly screening compounds on the immobilized modified targets on the resin). The batch format may be automated by integrating liquid handling robots, vacuum manifolds, and multi-well plate format injectors, chromatographic platforms and sample collectors.

Self-packed columns allow the users to make columns of custom designed size and capacity and to their own requirements. The use of self-packed columns is often for speciality resins that may not be available in pre-packed or batch format.

Pre-packed columns allow the users to purchase columns commercially packed or validated that have common parameters and functionality. The quality control is very high and the reproducibility from column to column is an asset. The ease of use and comprehensive data files accompanying the column make for simple and easy integration and use directly off the shelf. In addition, the systems are easy to adapt to a wide range of chromatographic platforms. Furthermore, the “bioprocess” validation that is possible with many columns ensures that the column may be used in drug production processes.

A powerful advantage to this method is the coupling of affinity purification to modification to on-column cleavage in an automated, systematic and reproducing manner on existing chromatographic platforms (ÄKTA™ or Ettan™ systems ex Amersham Biosciences of Uppsala, Sweden) producing a protein or polypeptide product that optionally has no affinity fusion tag following modification and cleavage. This has many advantages such as more ‘native’ like form (in the case of no fusion tag present) and isolation of modified target protein or polypeptide only, no contamination of fusion protein.

Secondly, when applied to recombinant proteins or polypeptides, produced in prokaryotic cells, it allows for the production of a protein or polypeptide close to the eukaryotic form: the modifications done to immobilized target protein or polypeptide produce eukaryotic-like modifications currently not possible to systematically control in vivo or under poor control and limited enrichment using in vitro modification systems.

Thirdly, a flexible method is provided which functions for a wide range of proteins or polypeptides, such as e.g. nuclear, cytosolic and membrane proteins, proteins differing in chemical and/or functional nature, enzymes, nucleic acid (RNA and DNA) binding proteins and complexes, immunoproteins, structural proteins and signaling proteins as well as corresponding polypeptides.

In view of the temperature dependence of the kinetics of the enzymatic reaction, prior art methods for enzymatic reactions are generally performed at temperatures around 37° C. A decrease of the temperature would result in an increase of the reaction time, which could become unacceptably long. On the other hand, at the temperature required by the prior art methods, the stability of the protein and the polypeptide is susceptible of being deteriorated. This may be due both to increased unfolding and misfolding of the protein and polypeptide with an increasing temperature and to an increased risk of degradation reaction to occur, e.g. as catalyzed by any trace contamination of proteases. An advantageous feature according to the present invention is that the entire process can be performed at an appropriately selected temperature, which may be held constant or varied as required. For example, the condensed phases may be maintained at ambient temperature (about 25° C.). More preferably, the method of the invention is performed at a temperature from 15° C. to as low 2° C., a most preferable temperature being 4° C. At this low temperature a slower reaction due to temperature dependence may be compensated for by repeatedly bringing the enzyme preparation into contact with the solid phase carrying the immobilized protein or polypeptide, such as by recirculating continuously a liquid flow containing the enzymatic preparation. By the inventive method the repeated circulation of the modification component, be it chemical or enzymatic, into contact with the protein or polypeptide immobilized on the solid phase will allow for a relatively short total time of modification even at a reaction temperature where, by the prior art methods, an unacceptably long duration of reaction period would be required.

As a further advantage of the low temperature of the modification reaction, a further enhanced specificity of reaction is obtained, e.g. due to the fact that the enzyme at a lower temperature is less susceptible of spurious reactions.

The method of the invention advantageously may be performed in an automated high-throughput experimental design, using chromatographic platforms and columns such as the ÄKTA™ and Ettan™ Platforms, from Amersham Biosciences of Uppsala, Sweden, but is not limited to the use on these specific platforms, or to the scale of these platforms.

The method, e.g. as performed on an ÄKTA platform for separation and modification, can be scaled linearly (approximately) from the microlitre (protein or polypeptide production measured in growth media) to 100's of litre scale. The method can also be applied using the Ettan platform for coupling of the modification to identification and characterization of modification by mass spectrometry. This may be a valuable tool e.g. when the downstream event is functional genomics, drug target screening applications or applications where highly specific data is required concerning the characterization of the modified target protein or polypeptide.

In relation to drug discovery and drug screening, the method of the invention provides a very important improvement. By use of a method of the invention the eukaryote protein or polypeptide of interest, e.g. associated to a disease, may be recombinantly produced in a suitable overexpressing host, such as a bacterial strain, whereafter it is precisely and selectively modified to similarity with the native eukaryote protein or polypeptide. This protein or polypeptide may be used, either in the immobilized state or after collecting it from the solid phase, for efficient screening of substances for use as potential drugs.

According to one aspect of the invention, in relation to potential drug discovery, proteins or polypeptides, post-translationally modified by a method according to the invention, may be used to raise antibodies for the use in treating diseases or in the case of profiling or detecting disease, or the onset of certain disease states.

Accordingly, a PTM biomolecule may be produced by modification of the corresponding non-PTM biomolecule, and the PTM biomolecule may the be used in generating or raising antibodies against that specific modification, relative to its unmodified parental biomolecule.

The antibodies may be used to treat diseases with specific antibody-antigen recognition and biological implications, or as detection or profiling screens to study the onset or possible onset of disease in a diagnostic and/or screening type approach.

In an advantageous embodiment, the solid phase is an affinity resin to which the target protein or polypeptide is immobilized by means of a fusion linker. This may be achieved by manipulating, at the gene level, a target protein or polypeptide by fusing it to another protein or peptide fragment, to obtain a so-called fusion protein or polypeptide. At the protein expression level, fusion proteins or polypeptides can have the advantage of providing a more favorable gene construct organization permitting higher levels of soluble protein or polypeptide to be expressed, and possibly reducing the propensity to drive the protein or polypeptide folding process towards creating inclusion bodies. However, an inherent problem with the fusion protein/peptide system is that the ‘tag’ is often difficult to remove. Specific proteases required to perform the cleavage reaction necessary to separate the fusion tag from a target protein have inherent difficulties manifesting themselves as: (i) non-specific proteolytic attack of the target protein; (ii) the need for elevated temperatures for efficient cleavage, often resulting in the denaturation or aggregation of the target protein; (iii) incomplete proteolytic processing resulting in partially cleaved target protein, thereby significantly reducing the yield and/or introducing heterogeneity to the purified protein; (iv) additional purification steps are necessary to separate the cleaved target protein from the fusion tag, deactivate and remove the processing protease and exchange or desalt buffer components.

Several fusion protein technologies are known and may be used, such as by use of:
Glutathione-S-Transferase GST (S. Markrides, Micro. Biol. Rev., 60 (1996) 512-538; J. Nilsson, S. Ståhl, J. Lundeberg, M. Uhlén and P.-Å. Nygren, Prot. Expr. Purif., 11 (1997) 1-16);
polyhistidine tags [E. Hochuli, W. Bannwarth, H. Döbeli, R. Gentz and D. Stüber, Bio/Technology, 6 (1988) 1321-1325; E. Hochuli, H. Döbeli and A. Schacher, J. Chromatogr., 411 (1987) 177-184);
FLAG-tags [B. L. Brizzard, R. G. Chubet, D. L. Vizzard, BioTechinques, 16 (1994) 730-734; T. P. Hopp, K. S. Prickett, V. L. Price, R. T. Libby, C. J. March, D. P. Cerretti, D. L. Urdal, and
P. J. Conlon, Bio/Technology, 6 (1988) 1204-1210; A. Knappik and A. Plückthun, BioTechniques, 17 (1994) 747-761];
thioredoxin [E. R. LaVallie, E. A. DiBlasio, S. Kovacic, K. L. Grant, P. F. Schendel and J. M. McCoy, Bio/Technology, 11 (1993) 187-193; Z. Lu, E. A. DiBlasio-Smith, K. L. Grant, N. W. Warne, E. R. LaVallie, L. A. Collins-Racie, M. T. Follettie, M. J. Williams and J. M. McCoy, J. Biol. Chem., 271 (1996) 5059-5065; D. L. Wilkinson, N. T. Ma, C. Haught and R. G. Harrison, Biotechnol. Prog., 11 (1995) 265-269.];
Protein A [J. Nilsson, P. Nilsson, Y. Williams, L. Pettersson, M. Uhlén and P.-Å. Nygren, Eur. J. Biochem, 224 (1994) 103-108; E. Samuelsson, T. Moks, B. Nilsson and M. Uhlén, Biochemistry, 33 (1994) 4207-4211; M. Uhlén, B. Nilsson, B. Guss, M. Lindberg, S. Gatenbeck and L. Philipson, Gene, 23 (1983) 369-378.];
Strep-tag [J. Nilsson, M. Larsson, S. Ståhl, P.-Å. Nygren and M. Uhlén M. J. Mol. Recognit., 9 (1996) 585-594; T. G. M. Schmidt and A. Skerra, Prot. Eng., 6 (1993) 109-122.]; and
Maltose-binding protein [H. Bedouelle and P. Duplay, Eur. J. Biochem., 171 (1988) 541-549; C. di Guan, P. Li, P. D. Riggs and H. Inouye, Gene, 67 (1988) 21-30; C. V. Maina, P. D. Riggs, A. G. Grandea III, B. E. Slatko, L. S. Moran, J. A. Tagliamonte, L. A. McReynolds and C. diGuan, Gene, 74 (1988) 365-373].
   Glutathione-S-Transferase (GST) Fusions

Currently available Glutathione-S-Transferase (GST) systems can be used with GST-affinity resins using coupled glutathione or glutathione derivatives as ligand and GST or GST derivatives for binding as a fusion-protein binding component.

Polylhistidine Tags

Currently available polyhistidine (or derivatives, e.g. His-Glu-His-Glu systems included) can be used in association with a number of types of immobilizing chelating resins, such as IMAC-chelating (ex Amersham Biosciences of Uppsala, Sweden), Ni-NTA (ex Qiagen), Talon (ex Clonetech), Tentatcle (ex Merck) etc.

Maltose Binding Protein (MBP) Fusions

Currently available MBP systems can be used, with MBP-affinity resins using coupled amylose (or derivatives, or other compatible sugars) and MBP (or derivatives) for binding as a fusion-protein binding component.

Immobilization of the target protein or polypeptide on the solid phase also may be achieved by use of currently available or custom designed affinity purification systems based on immuno-recognition, either by antibodies or antibody/antigen fragments bonded to suitable resins. The immobilization could be mediated by a specific and/or non-specific binder (molecule and/or moiety with some affinity for the target protein).

Still other means for immobilizing the protein or polypeptide may be used, provided they selectively bind the protein or polypeptide to be modified, possibly associated to a moiety capable of selective immobilization on the solid phase. Such may be a moiety attached chemically to the protein or polypeptide, e.g. by covalent binding, ionic binding etc, or may be a moiety such as a fusion tag. This immobilization moiety should bind sufficiently strongly both to the protein or polypeptide and the solid phase, to allow the protein or polypeptide to remain imrnobilized during the modifying reaction, but still in a reversible way to allow for subsequently collecting the modified protein or polypeptide, by breaking the bond between either the immobilization moiety and the solid phase or between protein or polypeptide and the immobilization moiety, or both. Suitable currently available examples are Heparin, Biotin/Streptavidin, nucleotide binding solid phases, as well as ion exchange resin.

Heparin Sepharose is an affinity chromatography resin. A Heparin Sepharose resin provides fast, preparative separations of proteins and other biomolecules based on their affinity for heparin. Heparin is a naturally occurring glycosaminoglycan, which is an effective affinity binding and cation ion exchange ligand for a wide range of biomolecules, including DNA binding proteins, coagulation factors and other plasma proteins, lipoproteins, protein synthesis factors, enzymes that act on nucleic acids and steroid receptors. By coupling heparin to Sepharose™ with a chemically optimized linkage, an excellent medium for affinity purification is provided.

Purified Streptavidin isolated from Streptomyces avidinii is immobilized on Sepharose™ beads. The immobilized streptavidin binds biotin and biotinylated substances and can be used for affinity chromatography applications. The interaction between streptavidin and biotin is very strong. It can be used in the purification of antigens, where biotinylated antibodies are incubated with antigen. The biotinylated antibody-antigen complex binds to HiTrap Streptavidin from which the antigen can be eluted. Another example is to utilize the interaction between 2-iminobiotin and streptavidin, eluting the bound substances at pH 4.

Nucleotide binding may be performed on Blue Sepharose 6, which is Cibacron Blue 3G coupled to Sepharose. A member of the BioProcess™ Media family. It is particularly suitable for the isolation and purification of albumin, interferon, a broad range of nucleotide requiring enzymes, α-macro-globulin, coagulation factors, and nucleic acid binding proteins.

Ion exchange (IEX) separates proteins with differences in charge to give a very high resolution separation with high sample loading capacity. The separation is based on the reversible interaction between a charged protein and an oppositely charged chromatographic medium. Proteins are immobilized, as they are loaded onto a column. Conditions are then altered so that the immobilized substances are eluted differentially. This elution is usually performed by increases in salt concentration or changes in pH. Changes are made stepwise or with a continuous gradient. Most commonly, samples are eluted with salt (NaCl), using a gradient elution. Target proteins are concentrated during binding and collected in a purified, concentrated form.

Hydrophobic interaction separates proteins with differences in hydrophobicity. The separation is based on the reversible interaction between a protein and the hydrophobic surface of a chromatographic medium. High ionic strength buffer enhances these interactions. Samples in high ionic strength solution (e.g. 1.5 M ammonium sulphate) are immobilized, as they are loaded onto a column. Conditions are then altered so that the immobilized substances are eluted differentially. Elution is usually performed by decreases in salt concentration. Changes are made stepwise or with a continuous decreasing salt gradient. Targets are concentrated during immobilization and collected in a purified, concentrated form. Other elution procedures include reducing eluent polarity (ethylene glycol gradient up to 50%), adding chaotropic species (urea, guanidine hydrochloride) or detergents, changing pH or temperature.

Reverse Phase chromatography separates proteins and peptides with differing hydrophobicity based on their reversible interaction with the hydrophobic surface of a chromatographic medium. Samples are immobilized, as they are loaded onto a column. Conditions are then altered so that the immobilized substances are eluted differentially. Due to the nature of the reversed phase matrices, immobilization is usually very strong and requires the use of organic solvents and other additives (ion pairing agents) for elution. Elution is usually performed by increases in organic solvent concentration, most commonly acetonitrile: Samples, which are concentrated during the binding and separation process, are collected in a purified, concentrated form.

According to one aspect of the invention, kits are provided. Kits according to the invention may include a number of suitable components, depending on the starting material, the desired modification(s), the purpose of the modified protein or polypeptide etc.

A kit according to the invention comprises components for modifying an immobilized protein or polypeptide, i.e. at least one chemically reactive system or at least one enzymatically reactive system, or a combination of both systems.

In relation to drug discovery and disease detection, profiling and treatment, a kit for detection of a post-translationally modified protein or polypeptide may comprise all the components for production of the post-translationally modified protein or polypeptide, or the post-translationally modified protein or polypeptide per se, and components necessary for raising antibodies against the modified protein or polypeptide. In addition, this applies to diagnostic assays and screens that may be used as analytical tools or in high-throughput screening applications.

A chemically reactive system, may comprise any number of reactive components suitable for performing a given modification reaction, e.g. one or several reagents as well as catalysts. The chemicals may have any suitable form, such as powder, liquid solution, emulsion, suspension etc.

A reactive system of the invention may include biological components to complete the reaction, such as biological membranes; isolated and reconstituted enzymes and cellular components (including (endogenous or supplemented) lipids, co-factors, salts, substrates).

Bioactive membranes and/or programmed lysates applicable in the method and kits according to the invention can be endogenous biological membranes or cellular solutions extracted from an organism, collected and used in an alternative system. These membranes and/or lysates are biologically active and perform as a function of their components, their organization, their stoichiometry, and primarily, their inherent function in their endogenous environment (a membrane or mulitmeric complexes). Additional factors include, unknown biologically relevant components that have not been identified or characterized. The isolation of these bioactive membranes and/or programmed lysates can also encompass the reconstitution of the active form of the membrane and/or cellular components by adding the active components and substances that provide the bioactive membrane and/or lysate with its active function.

The enzymatically reactive system comprises at least one enzyme capable of catalyzing a modification reaction and any auxiliary components necessary for the modification reaction to occur. The enzyme may be in any form suitable for the purpose of the invention. E.g. it may be a purified preparation to be stored at a temperature between −20° C. and −80° C., or a preparation in a suitable buffer, to be stored at a temperature preferably of about +4° C. The enzyme preparation may also be in a lyophilized form.

A kit according to the invention may also comprise components for immobilizing the protein or polypeptide on a solid phase.

Furthermore, a kit according to the invention may comprise components for freeing and collecting the modified protein or polypeptide from the solid phase.

In one embodiment, in addition to the components for modifying an immobilized protein or polypeptide, a kit according to the invention comprises: components for producing/obtaining a protein or polypeptide to be modified; components for immobilizing the protein or polypeptide on a solid phase; components for freeing and collecting the modified protein or polypeptide from the solid phase; and components for testing/operating/monitoring the process.

The components for obtaining a protein or polypeptide to be modified may comprise e.g. a vector coding for the protein, optionally an affinity fusion tag construct; a bacterial strain suitable for overexpressing the protein or polypeptide; reagents and substrates suitable for cultivating the bacteria and enhance overexpression.

The vector coding for a protein e.g. may be a plasmid vector, such as a pGEX vector or derivation thereof.

The bacterial strain suitable for overexpressing the protein may be e.g. BL21 of Eschierichia coli or a derivation thereof.

The reagents and substrates suitable for cultivating the bacteria and enhance overexpression may be e.g. growth media such as Luria Broth, inducing reagent (IPTG), antibiotics for antibiotic resistance and protease inhibitors.

The components for immobilizing the protein on a solid phase may comprise a suitable solid phase and a liquid phase comprising protein or polypeptide purification reagents.

The solid phase may be any solid phase capable of immobilizing selectively the protein or polypeptide of interest, e.g. affinity resins or ion exchange gels. Preferably the solid phase is an affinity resin, e.g. Glutathione Sepharose, HiTrap™ IMAC chelating, from Amersham Biosciences, or derivatives thereof.

The protein purification reagents may be e.g. buffers, such as PBS; reducing agent, such as DTT; elution competitors, such as reduced glutathione or imidazole; protease inhibitors; and detergents.

The components for modifying the immobilized protein may comprise one or several modifying enzymes, selected in accordance with the particular modification(s) one desire to perform, as well as any auxiliary components required or suitable for performing the modification reaction(s).

In the case of a kit comprising an enzymatic modification system, the choice of the enzyme of course is primordial for effecting the desired modification. It is within the knowledge of the person skilled in the art to select a proper enzyme for any particular modification reaction. E.g. to perform a phosphorylation, a Icinase is required, whereas to perform a glycosylation, a glycosylase is required. In the case of a kit comprising several enzymes, these may be provided separately or as a mixture, or as a combination of both.

For selection of suitable enzymatic modification components, reference may be made to web sites such as:

EXPASY-Post-Translational Modification Predictions

Swiss Institute of Bioinformatics (SIB) Appel R. D., Bairoch A., Hochstrasser D. F. A new generation of information retrieval tools for biologists: the example of the ExPASy WWW server. Trends Biochem. Sci. 19:258-260(1994). (http://www.expasy.ch/tools/#ptm).

PhosphoBase

Kreegipuu A, Blom N, Brunak S (1999), “PhosphoBase, a database of phosphorylation sites: release 2.0.”, Nucleic Acids Res 27(1):237-239 (http://www.cbs.dtu.dk/databases/PhosphoBase/).

O-GLYCBASE version 4.0:

Database of O-glycosylated proteins. Ramneek Gupta, Hanne Birch, Kristoffer Rapacki, Søren Brunak and Jan E. Hansen. Nucleic Acids Research, 27: 370-372, 1999. (http://www.cbs.dtu.dk/databases/OGLYCBASE/).

Protein Information Resource

A Division of National Biomedical Research Foundation Protein Information Resource, National Biomedical Research Foundation 3900 Reservoir Rd., NW, Washington, D.C. 20007, USA (http://pir.georgetown.edu/).

The auxiliary components required or suitable for performing the modification reaction(s) may comprise buffers, such as Tris-HCl; detergents; salts; reducing agents; required substrates/co-factors, e.g. ATP, phosphatidylserine; and protease inhibitors.

Herein below, a list of components, necessary (n) and optional (O), of some kits according to the invention, is provided:
Phosphorylation: kinase (n), buffers (O), substrates/co-factors (ATP; n), additives (O), salts (O);
Dephosphorylation: phosphatase (n), buffers (O), substrates/co-factors (ATP; n), additives (O), salts (O);
SUMOylation: carboxy-terminal hydrolase (O), activating enzyme (n), conjugating enzyme (n), ligating enzyme (n), buffers (O), substrates/co-factors (ATP; n), additives (O), salts (O);
Ubiquitinylation: carboxy-terminal hydrolase (O), activating enzyme (n), conjugating enzyme (n), ligating enzyme (n), buffers (O), substrates/co-factors (ATP; n), additives (O), salts (O); Carboxymethylation: carboxymethylase (n), buffers (O), substrates/co-factors (ATP; n), additives (O), salts (O);
Acetylation: acetyltransferase (n), buffers (O), substrates/co-factors (ATP; n), additives (O), salts (O);
Deacetylation: deacetyltransferase (n), buffers (O), substrates/co-factors (ATP; n), additives (O), salts (O);
Amidation: acyltransferase/amidase (n), buffers (O), substrates/co-factors (ATP; n), additives (O), salts (O);
Methylation: methyltransferase (n), buffers (O), substrates/co-factors (ATP; n), additives (O), salts (O);
Demethylation: carboxy-methyltransferase (n), buffers (O), substrates/co-factors (ATP; n), additives (O), salts (O);
Carboxylation: carboxylase (n), buffers (O), substrates/co-factors (ATP; n), additives (O), salts (O);
Carboxyamylation: carboxylase (n), buffers (O), substrates/co-factors (ATP; n), additives (O), salts (O);
Sulphation: sulphotransferases (n), buffers (O), substrates/co-factors (ATP; n), additives (O), salts (O);
Hydroxylation: hydroxylase (n), buffers (O), substrates/co-factors (ATP; n), additives (O), salts (O);
ADP-ribosylation: ADP-ribosylation factors (ARFs; n), buffers (O), substrates/co-factors (ATP; n), additives (O), salts (O);
Maturation: proteolytic processing proteases (n), buffers (O), substrates/co-factors (ATP; n), additives (O), salts (O);
Adenylation: adenylyl transferase (n), buffers (O), substrates/co-factors (ATP; n), additives (O), salts (O);
Glycosylation: glycosylase (n), buffers (O), substrates/co-factors (ATP; n), additives (O), salts (O); and
Prenylation: prenyltransferase (n), buffers (O), substrates/co-factors (ATP; n), additives (O), salts (O).

The components for freeing and collecting the modified protein from the solid phase may comprise proteolytic processing enzyme, such as PreScission™ protease or thrombin; cleavage reagents, such as buffers, e.g. Tris; salts; detergents; reducing agents and, if required, affinity resins to capture protease, e.g. HiTrap benzamidine column (sold by Amersham Biosciences), or derivatives thereof.

The components for testing/operating/monitoring the process may comprise hardware, such as a recirculation unit, e.g. ÄKTA platform recirculation connector kit; application notes/technical support, reagents for performing control reactions, e.g. to test fusion tag, such as GST-fusion or polyhistidine fusion that can be immobilized, modified and cleaved easily.

Some examples of kits are given herein below:

Kit for Phosphorylation of GST-Fusion Protein or Polyeptide

Such kit for example may contain any/all (or other) components:
(i) plasmid vector coding for affinity fusion tag construct (e.g. pGEX vector or derivation thereof);
(ii) bacterial strain (e.g. BL21, or derivation thereof);
(iii) affinity resin (e.g. Glutathione Sepharose (GSTrap) or derivatives thereof);
(iv) protein expression reagents (e.g. growth media (LB), inducing reagent (IPTG), antibiotics for antibiotic resistance, protease inhibitors)*;
(v) protein purification reagents (e.g. buffers (PBS), reducing agents (DTT), elution competitor (reduced glutathione), protease inhibitors, detergents);
(vi) modifying enzyme(s) (e.g. specific kinase (Protein Kinase C; PKC, or other kinase(s), or a mixture of known kinases, e.g. PKC+MAPK+casein kinase II+ . . . + . . . + . . . ) to allow for screening of potential phosphorylation sites on a systematic and controlled manner; programmed lysate may also be used (such as eukaryotic cell lysate that is capable of making the desired modification reaction));
(vii) master mix of modifying reaction components (e.g. 10× buffers (Tris-HCl), detergents, salts, reducing agents, required substrates/co-factors (ATP, phosphatidyl-serine), protease inhibitors);
(viii) proteolytic processing enzyme (e.g. PreScission protease);
(ix) cleavage reagents (e.g. 10× buffers (Tris), salts, detergents, reducing agents);
(x) available hardware upgrades (e.g. ÄKTA platform recirculation connector kit);
(xi) application note/technical support (e.g. working methods on available platforms, i.e., an off the shelf method to work for GST-fusion proteins on an ÄKTA platform in an automated manner);
(xii) control reactions (e.g. test GST-fusion that can be immobilized, modified and cleaved easily)*.
   *optional components.
   Kit for Phosphorylation of Polyhistidine Tagged Fusion Protein or Polypeptide

Such kit for example can contain any/all (or other) components:
i) plasmid vector coding for affinity fusion tag construct (e.g. his tagged vector or derivation thereof);
(ii) bacterial strain (e.g. BL21, or derivation thereof);
(iii) affinity resin (e.g. HiTrap IMAC chelating, or derivatives thereof);
(iv) protein expression reagents (e.g. growth media (LB), inducing reagent (IPTG), antibiotics for antibiotic resistance, protease inhibitors)*;
(v) protein purification reagents (e.g. buffers (PBS), reducing agents (DTT), elution competitor (imidazole), protease inhibitors, detergents);
(vi) modifying enzyme(s) (e.g. specific kinase (Protein Kinase C; PKC, or other kinase(s), or a mixture of known kinases, e.g. PKC+MAPK+casein kinase II+ . . . + . . . + . . . ) to allow for screening of potential phosphorylation sites on a systematic and controlled manner; programmed lysate may also be used (such as eukaryotic cell lysate that is capable of making the desired modification reaction));
(vii) master mix of modifying reaction components (e.g. 10× buffers (Tris-HCl), detergents, salts, reducing agents, required substrates/co-factors (ATP, phosphatidylserine), protease inhibitors);
(viii) proteolytic processing enzyme (e.g. Thrombin);
(ix) cleavage reagents (e.g. 10× buffers (Tris), salts, detergents, reducing agents);
(x) affinity resin to capture protease (e.g. HiTrap benzamidine column, or derivatives thereof);
(xi) available hardware upgrades (e.g. ÄKTA platform recirculation connector kit);
(xii) application note/technical support (e.g. working methods on available platforms, i.e., an off the shelf method to work for GST-fusion proteins on an ÄKTA platform in an automated manner);
(xiii) control reactions (e.g. test polyhistidine fusion that can be immobilized, modified and cleaved easily)*.
   *optional components.
   Kit for Glycosylation of GST-Fusion Protein or Polypeptide

Such kit for example can contain any/all (or other) components:
(i) plasmid vector coding for affinity fusion tag construct (e.g. pGEX vector or derivation thereof);
(ii) bacterial strain (e.g. BL21, or derivation thereof);
(iii) affinity resin (e.g. Glutathione Sepharose (GSTrap) or derivatives thereof);
(iv) protein expression reagents (e.g. growth media (LB), inducing reagent (IPTG), antibiotics for antibiotic resistance, protease inhibitors)*;
(v) protein purification reagents (e.g. buffers (PBS), reducing agents (DTT), elution competitor (reduced glutathione), protease inhibitors, detergents);
(vi) stable “programmed” lysates (e.g. microsomal system with all modifying enzymes (glycosylases) contained in biomembranes, or in solution, that can be incubated with the immobilized target protein modifying the target protein in a recirculated manner in-line, on-column in an automated manner; purified (possibly recombinant) components reconstituted in reaction mix may also be used);
(vii) master mix of modifying reaction components (e.g. 10× buffers (Tris-HCl), detergents, salts, reducing agents, required substrates/co-factors (ATP, phosphatidylserine), protease inhibitors);
(viii) proteolytic processing enzyme (e.g. PreScission protease);
(ix) cleavage reagents (e.g. 10× buffers (Tris), salts, detergents, reducing agents);
(x) available hardware upgrades (e.g. ÄKTA platform recirculation connector kit);
(xi) application note/technical support (e.g. working methods on available platforms, i.e., an off the shelf method to work for GST-fusion proteins on an ÄKTA platform in an automated manner);
(xii) control reactions (e.g. test GST-fusion that can be immobilized, modified and cleaved easily)*.
   *optional components.
   Kit for Amidation of GST-Fusion Peptide (C-Terminal Modification)

Such kit for example can contain any/all (or other) components:
(i) plasmid vector coding for affinity fusion tag construct (e.g. pGEX vector or derivation thereof);
(ii) bacterial strain (e.g. BL21, or derivation thereof);
(iii) affinity resin (e.g. Glutathione Sepharose (GSTrap) or derivatives thereof);
(iv) protein expression reagents (e.g. growth media (LB), inducing reagent (IPTG), antibiotics for antibiotic resistance, protease inhibitors)*;
(v) protein purification reagents (e.g. buffers (PBS), reducing agents (DTT), elution competitor (reduced glutathione), protease inhibitors, detergents);
(vi) modifying enzyme(s) (e.g. peptidylglycine alpha-amidating monooxygenase (PAM); programmed lysate (such as eukaryotic cell lysate that is capable of making the desired modification reaction) as well as chemical modification methods may also be used);
(vii) master mix of modifying reaction components (e.g. 10× buffers (Tris-HCl), detergents, salts, reducing agents, required substrates/co-factors (ATP, phosphatidylserine), protease inhibitors);
(viii) proteolytic processing enzyme (e.g. PreScission protease);
(ix) cleavage reagents (e.g. 10× buffers (Tris), salts, detergents, reducing agents);
(x) available hardware upgrades (e.g. ÄKTA platform recirculation connector kit);
(xi) application note/technical support (e.g. working methods on available platforms, i.e., an off the shelf method to work for GST-fusion proteins on an ÄKTA platform in an automated manner);
(xii) control reactions (e.g. test GST-fusion that can be immobilized, modified and cleaved easily)*.
   *optional components.
   Kit for Detection of a Post-Translationally Modified Protein or Polypeptide (PTM Biomolecule)

Such kit for example can contain any/all (or other) components:
(i) PTM biomolecule (ii) antibodies raised specifically against PTM biomolecule (in negative background on unmodified biomolecule)
(iii) master mix of antibody reaction components (e.g. 10× buffers (Tris-HCl), detergents, salts, reducing agents, required substrates/co-factors, protease inhibitors);
(iv) western blotting or ELISA detection systems
(v) available hardware upgrades
(vi) application note/technical support (e.g. working methods on available platforms, i.e., an off the shelf method to work for detection of PTM identified proteins on an existing platform in an automated manner);
(xii) control reactions (e.g. test PTM biomolecules with antibodies that can be detected and identified easily)*.
   *optional components.

The advantage provided by the various aspects of the invention resort from the above disclosure. The processes of the current state-of-the-art in producing modified proteins/peptides, have many drawbacks including inefficiency of production, expense, poor levels of purity, poor yield, extreme difficulties in isolation of natural product and heterogeneity. By the method of the invention, a systematic controlled approach is provided instead of relying solely on serendipitous interactions (programmed lysates, incubating target protein to be modified with eukaryotic lysates which contain endogenous modifying proteins and hope for target protein modifications after prolonged incubation periods) or multiple processing steps (in vitro purification of all the necessary components then reconstitute the entire mix to create the desired modification (low level) only to have to purify away all the reaction components in order to isolate the modified target protein). The integration of several pre-existing methods coupled to applications involving the systematic control of experimental design and conditions in an automated manner is entirely unique and novel. The current state-of-the-art is moving in the opposite direction towards biological modifications using selected eukaryotic cell lines where the final product is under the entire control of the organism. This often results in low levels of modifications, heterogeneous modifications, spurious modifications and the experimenter starts processing the product with compromised levels of material both in yield and quality. In contrast to this, by the method of the invention, the reactive system is driven and controlled entirely in a comprehensive systematic way that can be automated and can produce material that is simply not obtainable in reasonable yields in any currently existing system.

### EXAMPLES

To further elucidate the invention, two examples of post-translational modification of a protein by a method according to the invention are described herein below as Examples 1 and 2.

Further examples, i.e. Examples 3 and 4 respectively generally illustrate the scale-up of process steps of the inventive method. In these examples, the immobilized protein, which is a fusion protein, is treated with Pre-Scission protease to cleave off the fusion partner. However, it should be understood that any enzyme, supplemented with suitable components necessary for the reaction to occur, such as co-factors and substrates, could be used in place of the protease, according to the PTM which it is desired to perform.

In addition, Examples 3 and 4 illustrate the processing of a difficult target (TDG), making the application to novel experimentally challenging targets more feasible.

### Example 1

In the present example a human eukaryotic cell receptor protein, recombinantly produced in a bacterial host as a fusion protein, was phosphorylated by a method according to the invention.

Methodological and Experimental Summary

Immobilization, Purification, Modification and On-Column Cleavage of a Receptor Protein

An over-expressed GST:: fusion protein is: affinity bound and immobilized on Glutathione Sepharose resin; post-translationally modified in-line on-column by phosphorylation reaction with PKC and/or MAPK enzymes; efficiently cleaved on-column and eluted as a homogenous modified product. The purity of the eluted proteins is evaluated by SDS-PAGE and mass spectrometry.

After selecting for conditions which produce high levels of expressed GST::fusion protein, the GST::fusion protein containing lysate is clarified by subsequent centrifugation steps at ˜70,000×g and at ˜300,000×g. Following the ultracentrifugation step, the clarified lysate containing GST::fusion protein is loaded on a GSTrap™ FF column with PBS binding buffer and the unbound fraction passes through the column. PBS binding buffer is used to wash the GSTrap™ FF column until the absorbance baseline returns. At this point the buffer is exchanged to Phosphorylation buffer (PKC-pb or MAPK-pb) and equilibrated. The immobilized GST::fusion protein is then incubated with modifying enzyme (PKC and/or MAPK) injected and applied to the column in iterative steps. Single specific modifications, or multiple modifications are possible. The primary requisite is buffer exchange to optimum reaction buffers (PKC-pb and MAPK-pb) for each modification reaction. Following the modification reactions, the immobilized GST::fusion protein is washed by buffer exchange to cleavage buffer. The cleavage buffer acts primarily to equilibrate the GSTrap™ FF column prior to proteolytic cleavage by PreScission™ protease of the bound GST::fusion protein. This final buffer exchange plays an important role in downstream processes sensitive to phosphate salts, such as crystallization condition screening, and metal dependent biochemical assays. After the absorbance baseline returns, PreScission™ protease is loaded on the GSTrap™ FF column and the system is in a ‘closed’ position incubating for ˜12 h. Following incubation, the cleaved modified protein is eluted. After the absorbance baseline has returned, a 100% step gradient of reduced glutathione containing buffer, elution buffer, is introduced and acts as a competitor for GST binding sites. The bound GST::linker (proteolytic cleavage reaction product) and PreScission™ protease elute from the column. The column is regenerated after the absorbance baseline has returned and can be re-equilibrated with binding buffer for subsequent purification and on-column cleavage runs. In this example, this method can be scaled from small-scale (<1 ml cultures) to larger-scale (>20 litre cultures) in a linear manner and is directly proportional to materials and yield reproducing consistent levels of protein production. The overall purification, immobilization, modification and on-column cleavage strategy produces highly pure, enriched modified protein with a yield of ˜0.8 mg/litre culture in the purity range of 95% pure protein completed in a single chromatographic step.

GST::Fusion Protein Binding

The supernatant containing the GST::fusion protein fraction from the ultracentrifugation step (soluble or membrane containing fractions) was loaded on a GSTrap™ FF column (5 ml; Amersham Biosciences, Uppsala, Sweden), pre-equilibrated with PBS as binding buffer, at a flow rate of 1 ml/min. For all chromatographic steps, an ÄKTA™ explorer (Amersham Biosciences, Uppsala, Sweden) was used enclosed in a refrigeration unit cooled to 4° C. to ensure protein stability and reduce protein degradation. Chromatographic profiles monitor continuously absorbance (260 and 280 nm) and conductivity (mS/cm). The bound material was washed with PBS buffer until the absorbance baseline had returned. Once the baseline was stable, the buffer was exchanged with either PKC-Phosphorylation buffer (PCK-pb; 20 mM HEPES-KOH at pH 7.4; 10 mM MgCl₂; 1.7 mM CaCl₂; 600 μg/ml phosphatidyl serine; 1 mM DTT and 50 μM ATP) or MAPK-Phosphorylation buffer (MAPK-pb; 50 mM Tris-HCl at pH 8.0; 0.5 mM EDTA; 25 mM MgCl₂; 1 mM DTT; 50 μM ATP and 10% glycerol). Phosphorylation buffer equilibration was continued until stable absorbance baseline was achieved. At this stage, buffer flow was arrested.

In Vitro Phosphorylation of GST::Fusion Protein

For the in vitro phosphorylation reaction of immobilized GST::fusion protein (bound on the GSTrap column) with PKC and/or MAPK, the kinase was injected and incubated in-line on-column with 25 ng of PKC in PKC-pb or 25 ng of MAPK in MAPK-pb. All phosphorylation reactions were carried out at 4° C. for 30 minutes-12 hours, in the presence of protease and phosphatase inhibitors (1 μM okadaic acid; 200 μM Na₃VO₄). For iterative modification reactions, the immobilized fusion protein was washed with the subsequent buffer and reaction components prior to injection and incubation with modifying enzyme. This step can be cycled until the desired modifications using defined enzymes reach completion. The addition of the modifying enzyme in appropriate buffer component system can be applied to the column in a tightly controlled and monitored manner using a closed recirculation loop (P-950 pump connected to sample/super loop). This recirculating modifying enzyme method allows for multiple reaction rounds, decreases overall reaction time, decreases modifying enzyme concentrations necessary to complete the reaction and increases the population of modified target protein.

On-Column Cleavage Reaction

This method utilizes the technologies of a GST::fusion protein linked with an infrequently biologically occurring proteolytic cleavage site. In conjunction with glutathione affinity columns and a highly specific engineered protease to purify desired target proteins in high yields with high levels of enrichment. PreScission protease (Amersham Biosciences, Uppsala, Sweden) is a genetically engineered fusion protein consisting of GST fused to a modified human rhinovirus 3C protease. PreScission protease (2 Units enzyme/100 μg of bound fusion GST::fusion protein) was diluted in Cleavage buffer equal to 90% of the volume of the GSTrap™ FF column and injected into the column at an increased flow rate of ˜5-7 ml/min. Following injection, the column was placed in a closed flow status and the system was incubated on-line for 12-16 h at 4° C.

Elution of ‘Native’ Protein

An auxiliary GSTrap™ FF column (1 ml) pre-equilibrated with Cleavage buffer was connected downstream of the primary cleavage reaction column in-line with the fraction collector. Cleaved modified target protein elution occurs immediately upon flow start-up with Cleavage buffer at ˜1 mmin. Following cleaved target protein elution and the return of the absorbance baseline, the GST-affinity peak was eluted with Elution buffer (50 mM Tris-HCl at pH 8.0 and 10 mM reduced glutathione) in a full step gradient (100% elution buffer).

Column Regeneration/Equilibration

Following the elution of the target protein and the GST-affinity proteins, the column can be equilibrated for subsequent purification runs. Column equilibration is completed by flushing the column with 3 column volumes of Milli-Q water followed by 3 column volumes of PBS Binding buffer. This regeneration stage is important for the throughput of the protein production process and allows for multiple runs to be completed in series.

Mass Spectrometry

Purified fusion protein was phosphorylated by PKC (Promega) according to manufactures instructions and subjected to SDS-PAGE. Samples were prepared according to Shevchenko et al. (Andrej Shevchenko, Igor Chemushevich, Matthias Wilm, and Matthias Mann, Methods in Molecular Biology, vol. 146: Protein and Peptide Analysis: New Mass Spectrometric Applications, Ed.: J. R. Chapman, Humana Press Inc., Totowa, N.J.). After tryptic digest of phosphorylated and control samples, peptides were extracted and analyzed by matrix-assisted laser desorption/ionization mass spectrometry using Voyager Biospectrometry Workstation with Delayed Extraction Technology, PerSeptive Biosystems, Inc. Obtained data was analyzed using Moverz software (Proteometrics, LLC). The mass spectrometric analysis showed that GST:: fusion protein had been post-translationally modified by PKC at a specific position.

### Example 2

In this example, a human developmental regulatory protein was immobilized and purified as a polyhistidine tagged fusion protein and submitted to two independent phosphorylation modifications.

The target Protein is 58 kDa from Homo sapiens and is designated p58 in this study. Parental vector encodes for p58 gene product in pBluescript vector isolated from cDNA library.

Over-expression vector sub-cloned into N-terminal His-tag pQE80 (His₆ₓ::p58).

Post-translational phosphorylation modifications of p58 with Protein Kinase C (PKC) and/or Casein Kinase 2 (CK-II).

Sub-Cloning

The cDNA encoding for p58 was excised from the pBluescript vector and ligated into a pQE80 vector creating p58-Q80. The p58 sub-clone was transformed into an E. coli cloning cell line MC1061. The plasmid construct was amplified and purified with DNA minipreps and p58-Q80 plasmid was transformed into the over-expression E. coli cell line M15.

Culturing

Overnight cultures of p58-Q80 transformed M15 bacteria were made by inoculating 5 ml of LB-media supplemented with Carbenicillin (100 μg/ml) and Kanamycin (50 μg/ml) with a single colony selected from the LB-agar plates. For the over-expression trials, 100 μl of overnight culture inoculated 100 ml LB-media supplemented with Carbenicillin (100 μg/ml) and Kanamycin (50 μg/ml). Over-expression trials controlled variables such as: temperature (30° C. and 37° C.); induction time (OD₆₀₀≈0.6 and OD₆₀₀≈1.0); induction concentration, IPTG (isopropyl-thio-β-D-galactoside) final concentrations (0.1 mM and 1.0 mM); and growth time, where growth curves were recorded observing for maximum amount of soluble over-expressed His₆ₓ::p58 target protein levels.
Over-expression assays grew overnight cultures at 18-37° C., containing antibiotics (100 μg/ml Carbenicillin) from fresh transformants in LB-Miller or 2×YT Medium.
Inoculate large cultures with 1/1000 inoculant from overnight cultures. LB-Miller or 2×YT Medium containing antibiotics (100 μg/ml Carbenicillin, 18-37° C.).
Grow the cells at 18-37° C., shaking at 244 rpm until OD₆₀₀≈0.6-1.0.
Induce the protein synthesis with 0.1-1.0 mM IPTG (final concentration) and let grow for 3-4 hours.
Harvest the cells in IL centrifuge cylinders and centrifuge at 5000×g at 4° C. for 30 minutes.
Wash the cell pellet and resuspended the cells in TB buffer (9.1 mM HEPES, 55 mM MnCl₂, 15 mM CaCl₂, 250 mM KCl adjusted to pH 6.7).
Cells are then pooled and centrifuged in centrifuge tubes, spun at 4000×g for 5 minutes at 4° C.
   Cell Lysis
Cells were lysed by resuspending the pellet in Lysis buffer (50 mM Tris-HCl pH 8.0, 100 mM NaCl and 2% glycerol, supplemented with: 50 mg of Lysozyme, EDTA-free Complete Protease Inhibitor Cocktail, DNase 1 and 10 mM MgCl₂) on ice. The volume is approximately 10 ml for a 500 ml culture, 40 ml for a 2 l culture and 120 ml for a 5 l culture.
Lysis is performed in sealed centrifuge tubes to allow for proper mixing
After pellet is homogenously resuspended in solution, freeze pellet in N₂ (1) until pellet is completely frozen, then rapidly transfer the tube to 20° C. water bath. Gently shake the tube to thaw the frozen lysate evenly. Repeat this freeze/thaw step three times.
After cell lysate is homogenously thawed after the third freeze/thaw step, centrifuge lysate at 180,000×g for 30 minutes at 4° C.
Remove supernatant and place clarified lysate in ultracentrifugation tubes. Centrifuge at 300,000×g for 90 minutes at 4° C.
   Immobilization
The supernatant containing the His₆ₓ::p58 protein fraction from the ultracentrifugation step was loaded on a HiTrap™ IMAC affinity column (5 ml; Amersham Biosciences, Uppsala, Sweden), pre-equilibrated with Binding buffer (50 mM Tris-HCl pH 8.0, 100 mM NaCl and 2% glycerol), at a flow rate of 1 ml/min. For all chromatographic steps, an ÄKTAT™ explorer (Amersham Biosciences, Uppsala, Sweden) was used enclosed in a refiigeration unit cooled to 4° C. to ensure protein stability and reduce protein degradation. Chromatographic profiles monitor continuously absorbance (260 and 280 nm) and conductivity (mS/cm).
Pre-equilibrate the IMAC affinity column (charged with Co²⁺) with Binding buffer.
Remove the ultracentrifugation supernatant and apply it to the pre-equilibrated affinity column.
p58 protein is immobilized on the affinity column
Immobilized p58 is gently washed with ˜10-20 column volumes of Binding buffer.
Bound sample is stringently washed with ˜10-20 column volumes of Wash buffer (50 mM Tris-HCl pH 8.0, 100 mM NaCl, 2% glycerol and 10 mM imidazole).
   In-Line on-Column Phosphorylation with PKC
Phosphorylation reaction of immobilized p58 fusion protein (bound on the Co2+ affinity column) with PKC.
The kinase was injected and incubated in-line on-column with 25 ng of PKC in PKC-Phosphorylation buffer (PCK-pb; 20 mM HEPES-KOH at pH 7.4; 10 mM MgCl₂; 1.7 mM CaCl₂; 600 μg/ml phosphatidyl serine; 1 mM DTT and 50 UM ATP)
All phosphorylation reactions were carried out at 4° C. for 30 minutes-12 hours, in the presence of protease and phosphatase inhibitors (Complete Protease Inhibitor (Roche); 1 μM okadaic acid; 200 μM Na₃VO₄).
The modifying enzyme in appropriate buffer component system is applied to the colurnn in a tightly controlled and monitored manner using a closed recirculation loop (P-950 pump connected to sample/super loop).
This recirculating modifying enzyme method allows for multiple reaction rounds, decreases overall reaction time, decreases modifying enzyme concentrations necessary to complete the reaction and increases the population of modified target protein.
   In-Line on-Columnn Phosphorylation with CK-II
Phosphorylation reaction of immobilized p58 fusion protein (bound on the Co2+ affinity column) with CK-II.

The kinase was injected and incubated in-line on-column with 25 ng of CK-II in CK-II Phosphorylation buffer (CK-II-pb; 25 mM Tris-HCl at pH 7.4; 1 mM EDTA, 1 mM DTT, 200 mM NaCl, 2% glycerol, 10 mM MgCl₂; 1.7 mM CaCl₂; 600 μg/ml phosphatidyl serine; 1 mM DTT and 50 μM ATP).
All phosphorylation reactions were carried out at 4° C. for 30 minutes-12 hours, in the presence of protease and phosphatase inhibitors (Complete Protease Inhibitor (Roche); 1 μM okadaic acid; 200 μM Na₃VO₄).

The modifying enzyme in appropriate buffer component system is applied to the column in a tightly controlled and monitored manner using a closed recirculation loop (P-950 pump connected to sample/super loop).
This recirculating modifying enzyme method allows for multiple reaction rounds, decreases overall reaction time, decreases modifying enzyme concentrations necessary to complete the reaction and increases the population of modified target protein.
   Modified Target Protein Elution
Immobilized modified p58 is eluted with ˜4-10 column volumes of Elution buffer (50 mM Tris-HCl pH 8.0, 100 mM NaCl, 2% glycerol and 200 mM imidazole).
The eluted p58-containing fraction is then concentrated (either by centrifugal methods or a with a pressurized ultra-filtration device).
   Analysis

Several different methods of analysis of modified p58 were performed for each immobilization, modification and purification process: sodium dodecyl sulphate polyacrylamide amide gel electrophoresis (SDS-PAGE) denaturing gel; native PAGE; Western blotting using different antibodies raised against His6x and p58; and mass spectrometry.

I. SDS-PAGE

The modified p58 protein purity and modification was analyzed by SDS-PAGE (3.5% stacking gel and 12% separation gel). Samples were mixed with 2×Fling-and-Gregerson sample buffer (55 mM Tris-HCl pH 6.8, 2% SDS, 7% Glycerol, 4% β-mercaptoethanol and 0.01% Bromophenol blue), boiled for 7 minutes at 95° C. and separated electrophoretically on the denaturing SDS-PAGE. The gel was run at 120V for approximately 1 hour and visualized with Coomassie Brilliant Blue staining.

II. Native-PAGE

Non-reducing native-PAGE was performed on modified p58 using the same buffers as with the SDS-PAGE, but without the denaturing reagent SDS, reducing agent β-mercaptoethanol and without boiling the samples. Electrophoreses was run at 120V for 2 hours, in the same manner as SDS-PAGE and visualized with Coomassie Brilliant Blue staining.

III. Immunoblotting

Western blots were made according to two different protocols. One was prepared by electro-blotting the protein onto nitrocellulose membrane for 1 hour at 100 V and blocking the membrane with 1-5% BSA prior to incubating with the primary antibody. The other protocol blocked the membrane with low-fat non-dairy milk (LFNDM). Several antibodies were assayed; anti-p58 (dilution 1:2,000) directed against p58 and anti-RGS-His6ₓ (dilution (1:2,000) directed against RGS-His6ₓ sequences. For secondary antibodies, anti-mouse IgG (dilution 1:30,000) and goat anti-rabbit (dilution 1:4,000) were used.

Mass Spectrometry

Purified p58 was phosphorylated by PKC and/or CK-II and subjected to SDS-PAGE. Samples were prepared according to Schevchenko et al. 2000. After tryptic digest of phosphorylated and control samples, peptides were extracted and analysed by matrix-assisted laser desorption/ionization mass spectrometry using Voyager Biospectrometry Workstation with Delayed Extraction Technology (PerSeptive Biosystems, Inc). Obtained data were analysed using Moverz software (Proteometrics, LLC).

### Conclusions

The p58 protein was produced as over-expressed recombinant material and subjected to tightly controlled and systematic immobilization, modification and purification process producing a biologically relevant phosphorylated species. Previously, using currently available technologies, a comprehensive study of this modified p58 species would not be possible at this level of purity and scale in an efficient manner. The final product was highly enriched in specifically phosphorylated p58 as defined by SDS-PAGE, Native-PAGE, Immunoblotting and Mass Spectrometry. Currently, the modified p58 protein is being used for functional and structural studies as well as in the drug discovery process.

### Example 3

Purification of the HNF4α LBD using On-Column Cleavage Strategy and GSTPrep 16/10 Column

Bacterial Growth and Protein Expression.

Rat HNF4α ligand binding domain (LBD, aminoacids 133-368) was expressed as a fusion protein with GST using pGEX-6p expression plasmid and BL21 C+E. coli host. Four liters of LB medium supplemented with 5% sucrose, ampicilin (100 μg/ml) and IPTG (0.1 mM) were inoculated with overnight bacterial culture to 0.1 OD₆₀₀ₙₘ. Bacteria were grown in 2.5 l Tunair flasks (Shelton Scientific, Shelton, Conn., USA) at 180 rpm, 18° C. for 17 hours. Cells were collected by centrifugation (Beckman J L A 8.1000, 20 min., 12,000 g) and washed in PBS buffer (140 mM NaCl, 2.7 mM KCl, 10 mM Na₂HPO₄, 1.8 mM KH₂PO₄ adjusted to pH 7.4).

Lysis and Lysate Clearance.

Bacterial pellet was resuspended in PBS supplemented with 1 mg/ml lysozyme, 10 U/ml DNase, 5 mM MgCl₂. After 30 minutes of incubation on ice, bacteria were subjected to sonication (12 min total, pulse 1 sec:2 sec=on:off) with constant stirring on ice. Bacterial lysate was clarified by centrifugation at 50,000×g, 4° C. for 30 minutes (Beckman, Type 45 Ti) and subsequent ultracentrifugation at 180,000×g, 4° C. for 1 hour (Beckman, Type 60 Ti).

Chromatography.

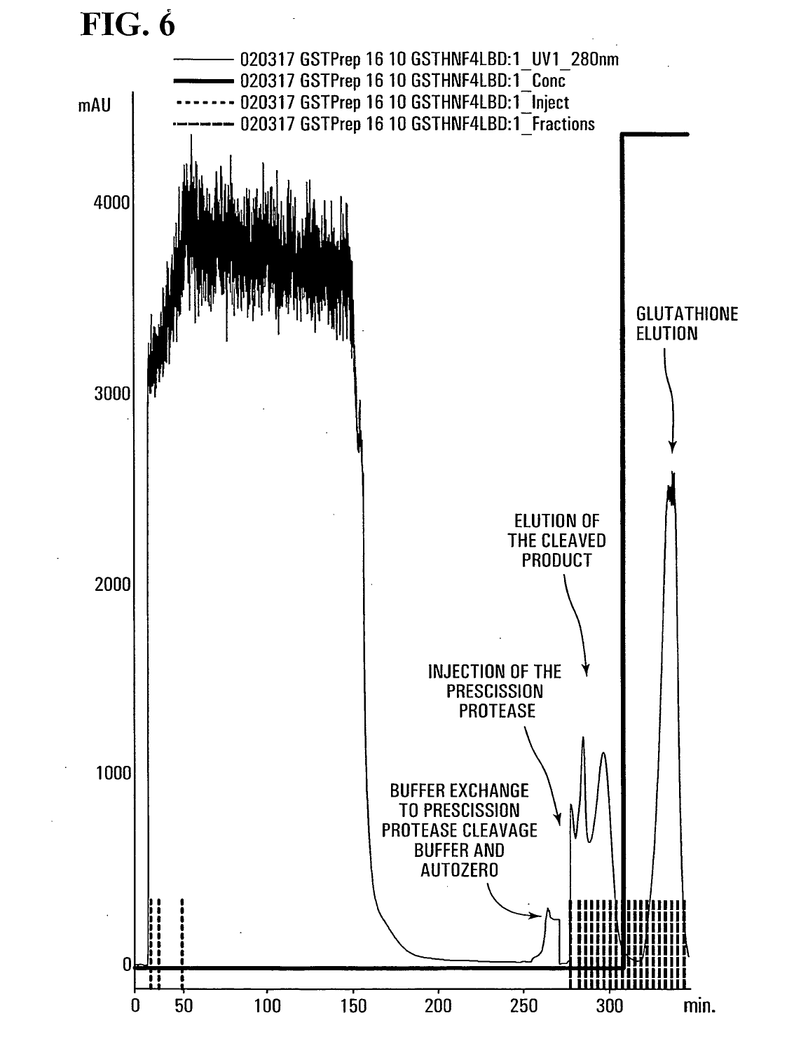

Clarified bacterial lysate (120 ml) was applied on the GSTPrep 16/10 column by the sample pump in the direct load mode. Column was extensively washed with PBS and buffer was changed for PreScission Protease cleavage buffer. For on-column cleavage, the column was disconnected from the system, 7 ml (⅓ of the column volume) of the cleavage buffer (50 mM Tris-HCl pH 8.0, 150 mM NaCl, 1 mM EDTA, 1 mM DTT) containing PreScission Protease (160 U) was injected on the column and incubated at 10° C. overnight. Cleaved product was collected in 22 ml of the cleavage buffer using GSTrap 1 ml auxiliary column downstream of the GSPrep 16/10 to eliminate GST and PreScission Protease leakage. The uncleaved protein was eluted by GSH elution buffer (50 mM Tris-HCl pH 8.0, 10 mM reduced glutathione) In FIG. 6, A corresponds to elution with PBS, PreScission Protease cleavage buffer; B corresponds to elution with the Glutathione elution buffer. Flow rate: 2 ml/min.

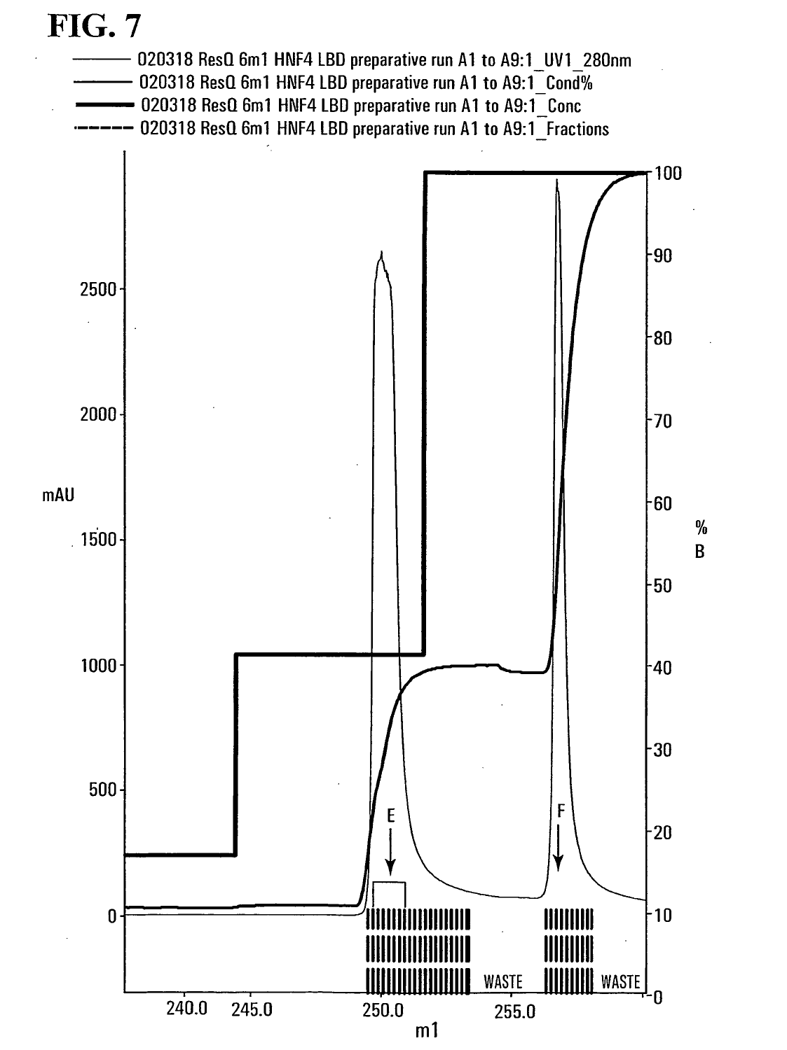

Protein sample from the on-column cleavage was injected on the Resource Q 6 ml column preequilibrated in 20 mM Tris-Cl pH 8.0, 50 mM NaCl. Unbound protein was washed by 150 mM NaCl until reaching baseline and HNF4α LBD was collected by 400 mM NaCl step in small fraction volumes (FIG. 7). This procedure both partially purified and concentrated target protein for the last purification step. In FIG. 7, A corresponds to elution with 20 mM Tris-Cl pH 8.0 and B corresponds to elution with 20 mM Tris-Cl pH 8.0, 1M NaCl. Flow rate: 3 ml/min.

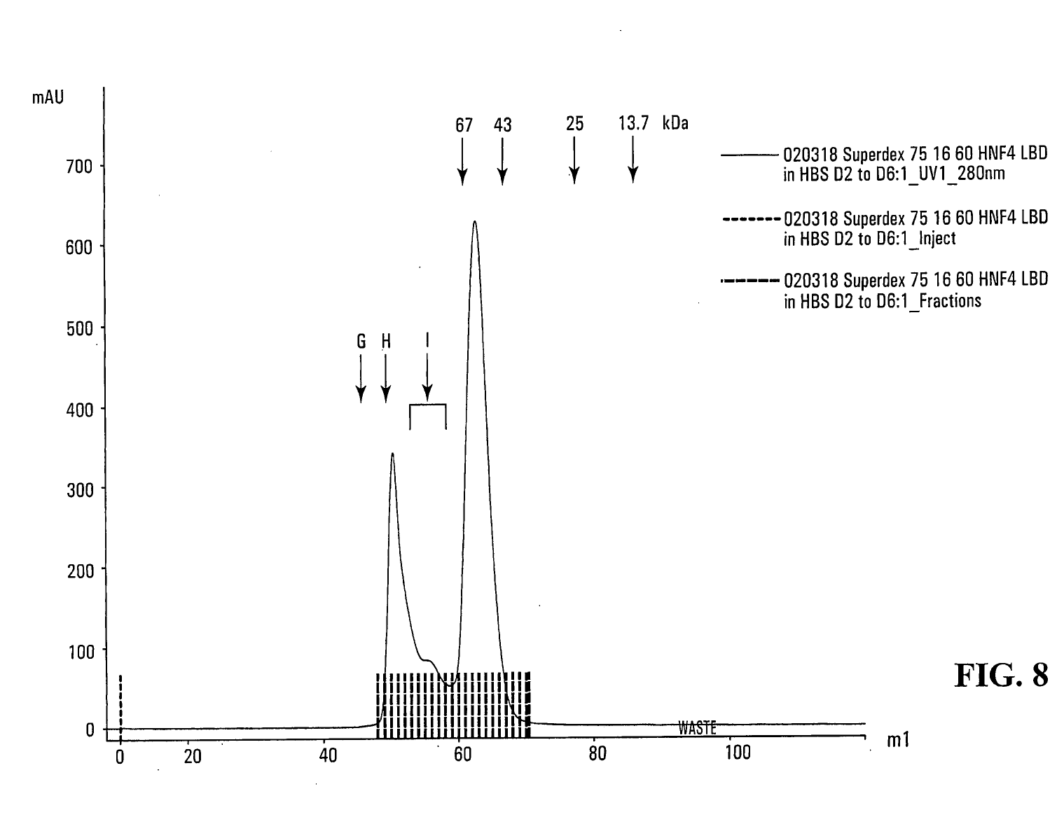

Fractions from the Resource Q column with highest protein concentration were pooled (0.8 ml) and injected on Superdex 75 16/60 column equilibrated in elution buffer (20 mM HEPES pH 7.4, 140 mM NaCl). The sample was fractionated at flow rate 0.25 ml/min overnight with automatic fractionation at 5 nAU level (FIG. 8). Target protein was eluted in the peak with apparent molecular weight 58.5 kDa, clearly separated from aggregated and/or complexed HNF4α LBD with bacterial chaperones. Molecular weight corresponds to LBD dimer (57.8 kDa) which is in agreement with the dimerisation properties of HNF4.

In FIG. 8, the arrows indicate elution volumes of the standard proteins from the Gel Filtration LMW Calibration Kit. Fractions analyzed by SDS-PAGE are indicated by capital letters.

HNF4 LBD was collected in 7 ml and concentrated using Microsep 10.000 MWCO (Pall Filtron) in gel filtration elution buffer to final volume 400 μl and protein concentration 12 mg/ml.

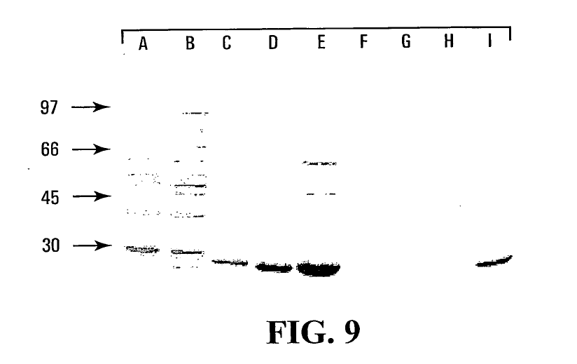

The protein samples from rat HNF4α LBD purification were analysed by SDS-PAGE. The results are represented in FIG. 9, wherein (A) corresponds to a bacterial lysate; (B) corresponds to the flow-through GSTPrep 16/10 column, (C) corresponds to on-column cleaved product, (D) corresponds to glutathione elution, (E) corresponds to concentrated protein by 40% B step on Resource Q 6 ml column, (F) corresponds to 100% B wash from Resource Q 6 m] column, (G,H,I) fractions from HiLoad 16/60 Superdex 75 prep column.

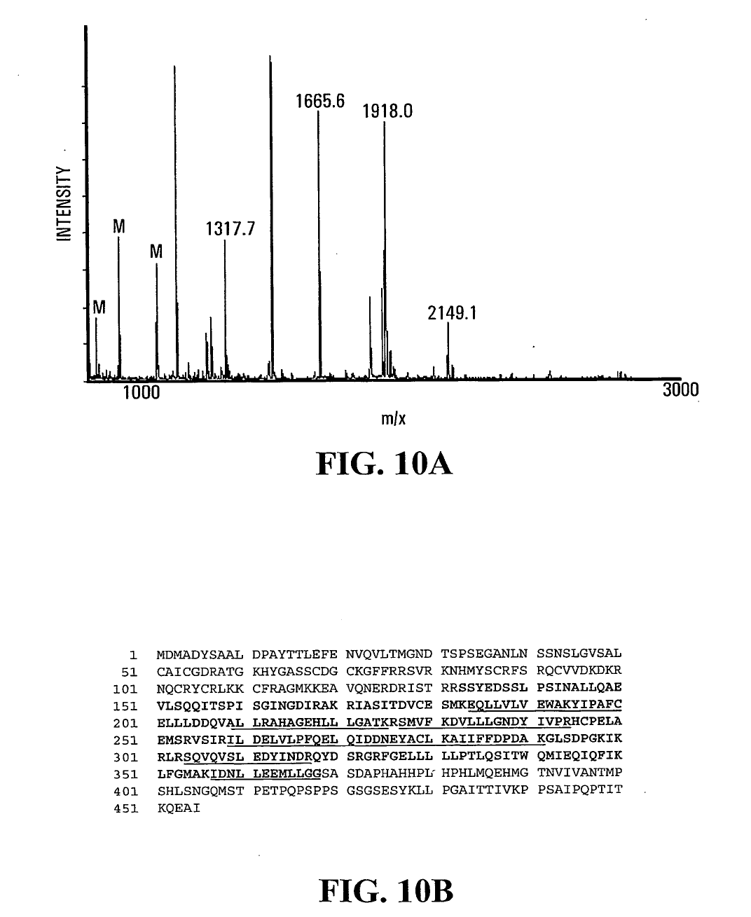

The purified rat HNF4α LBD was identified by MALDI-TOF MS. FIG. 10A represents the m/z spectrum of the peptides from tryptic digest. Peptide peaks corresponding to expected signals from rat HNF4α LBD are labelled with their molecular weight. M—matrix peaks.

FIG. 10B gives the full length sequence of the rat HNF4α protein. In bold, target protein corresponding to lig and binding domain is shown. Peptides identified by MALDI-TOF MS are underlined.

### Example 4

Purification of the Tymidine DNA Glycosylase (TDG) Using On-Column Cleavage Strategy and GSTPrep 16/10 Column

Bacterial Growth and Protein Expression

Mouse TDG was expressed as a fusion protein with GST using pGEX-6p expression plasmid and BL21 C+E. coli host. Eight liters of LB medium supplemented with ampicilin (100 μg/ml) were inoculated by 3% overnight bacterial culture. Bacteria were grown in 2 l E-flasks at 180 rpm, 37° C. After 2 hours temperature was lowered to 25° C. and incubation continued for additional one hour. Protein expression was induced by adding IPTG to final concentration 0.1 mM and the culture was incubated at 25° C. for four hours. Cells were collected by centrifugation (Beckman JLA 8.1000, 20 min., 12.000 g) and washed in PBS buffer (140 mM NaCl, 2.7 mM KCl, 10 mM Na₂HPO₄, 1.8 mM KH₂PO₄ adjusted to pH 7.4).

Lysis and Lysate Clearance.

Bacterial pellet was resuspended in PBS supplemented with 1 mg/ml lysozyme, 10 U/ml DNase, 5 mM MgCl₂. After 30 minutes of incubation on ice, bacteria were subjected to sonication (12 min total, pulse 1 sec: 2 sec=on: off) with constant stirring on ice. Bacterial lysate was clarified by centrifugation at 50.000×g, 4° C. for 30 minutes (Beckman, Type 45 Ti) and subsequent ultracentrifugation at 180.000×g, 4° C. for 1 hour (Beckman, Type 60 Ti).

Chromatography.

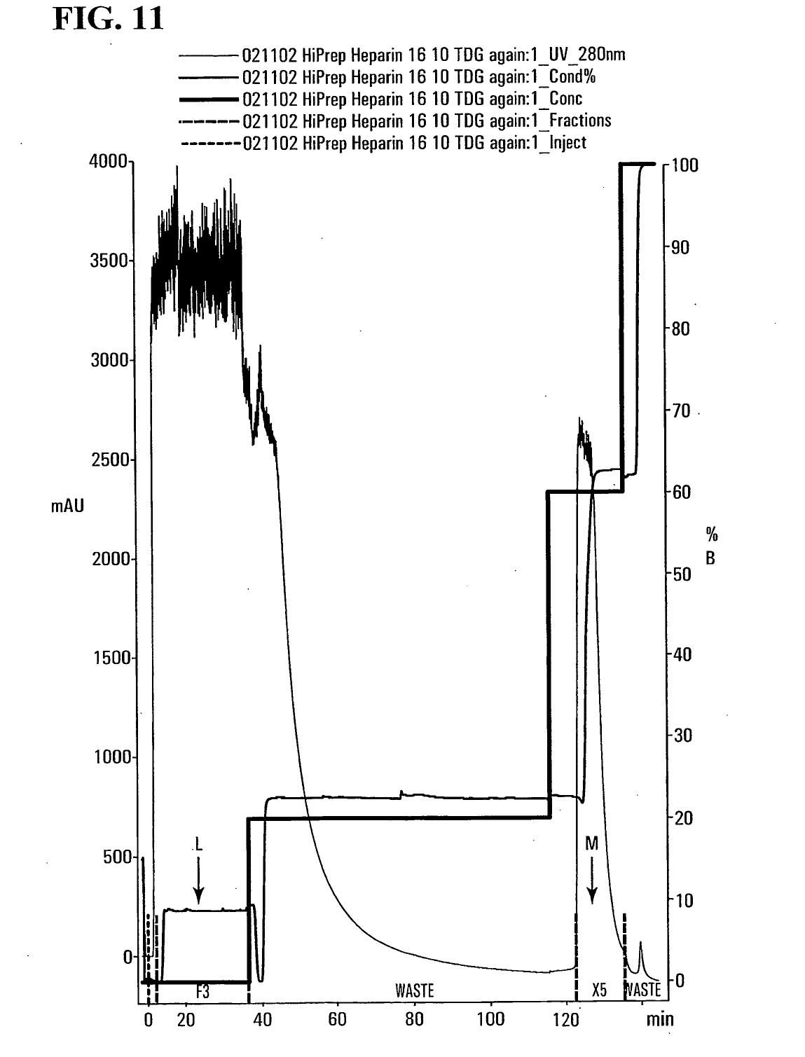

Clarified bacterial lysate (80 ml) was applied on HiPrep Heparin 16/10 column by a sample pump in the direct load mode. Column was extensively washed with 20% B and target protein was eluted by 60% B step (FIG. 11). Due to high target protein content in bacterial lysate, capture on HiPrep Heparin 16/10 column was repeated twice.

In FIG. 11, representing the purification of the mouse TDG on HiPrep Heparin 16/10 column, A stands for elution by PBS and B stands for elution by PBS, 1M NaCl. The flow rate was 5 ml/min.

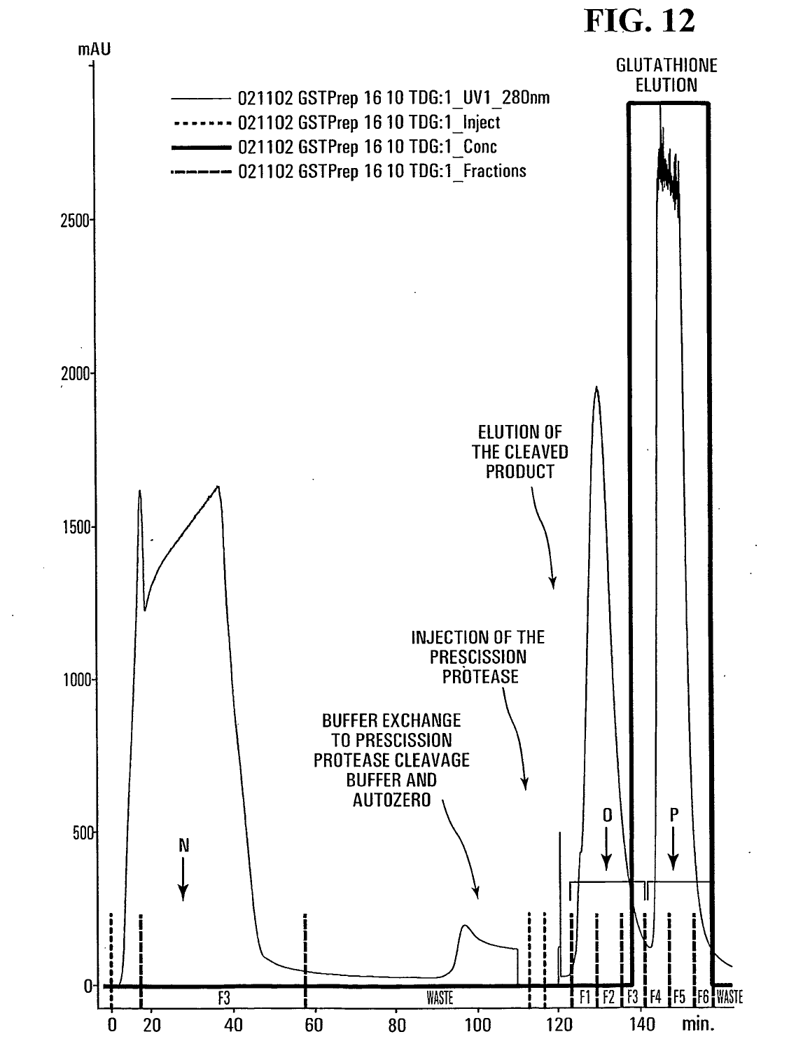

Eluted protein fractions were pooled and applied on GSTPrep 16/10 column. The column was washed by PBS buffer and buffer was changed for Cleavage buffer pH 8.8 (20 mM Tris-Cl pH 8.8, 50 mM NaCl, 1 mM EDTA, 1 mM DTT). Even though pH value of this buffer is higher than recommended for PreScission Protease, tests on parallel GSTrap 5 ml columins showed no difference in cleavage efficiency of GST::TDG when buffers of pH 8.0 and pH 8.8 were compared (data not shown). PreScission Protease (400 U) in 7 ml of Cleavage buffer pH 8.8 was injected on the GSTPrep 16/10 column by the sample pump in the direct load mode, taking in consideration dead volume of the pump and sample tubing (about 2.5 ml). After overnight incubation at 10° C., cleaved product was collected in 36 ml of the Cleavage buffer pH 8.8 using GSTrap 5 ml auxiliary column downstream of the GSTPrep 16/10 to eliminate GST and PreScission Protease leakage. The uncleaved protein was eluted by GSH elution buffer (50 mM Tris-HCl pH 8.0, 10 mM reduced glutathione) (FIG. 12).

In FIG. 12, representing purification of the mouse GST::TDG fusion protein on the GSTPrep 16/10 column, A stands for elution by PBS, Cleavage buffer pH 8.8; and B stands for elution by glutathione elution buffer. Flow rate: 2 ml/min.

Protein from the on-column cleavage was applied on Resource Q 6 ml column equilibrated in 5% B (A: 20 mM Tris-Cl pH 8.8, B: 20 mM Tris-Cl pH 8.8, 1M NaCl), washed by 10% B and 10%-25% B gradient in 40 column volumes was applied. Fractions from the major peak were collected, diluted with buffer A and re-applied on the same column in order to concentrate the sample by 40% B step prior to gel filtration (data not shown).

Half of the concentrated TDG from Resource Q 6 ml column (2 ml) was injected on HiLoad 16/60 Superdex 200 prep grade column and fractionated in buffer containing 20 mM Tris-Cl pH 8.0, 150 mM NaCl, 1 mM DTT and 0.1 mM EDTA at 1 ml/min.

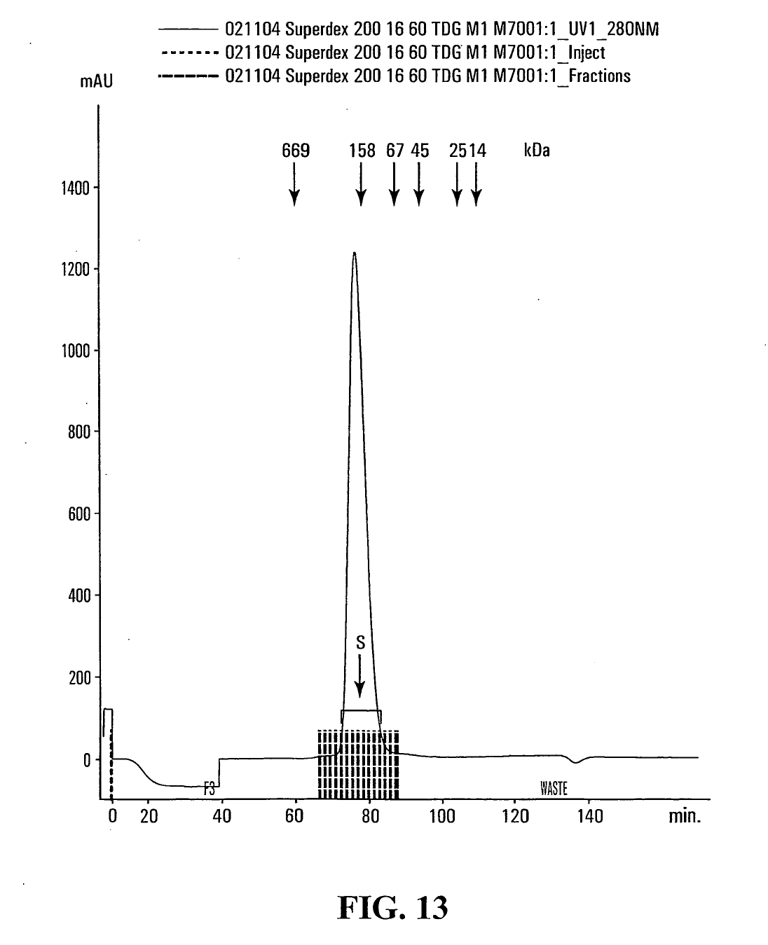

In FIG. 13, representing the purification of mouse TDG on HiLoad 16/60 Superdex 200 prep grade column, the arrows indicate elution volumes of the standard proteins from the Gel Filtration LMW and HMW Calibration Kits.

Fractions from the major peak were concentrated to volume 300 μl and concentration 22 mg/ml. Protein sample was analyzed by MALDI-TOF/MS in order to confirm identity of the purified protein.

Calculated Mw: 178 kDa (4×44 kDa=176 kDa)

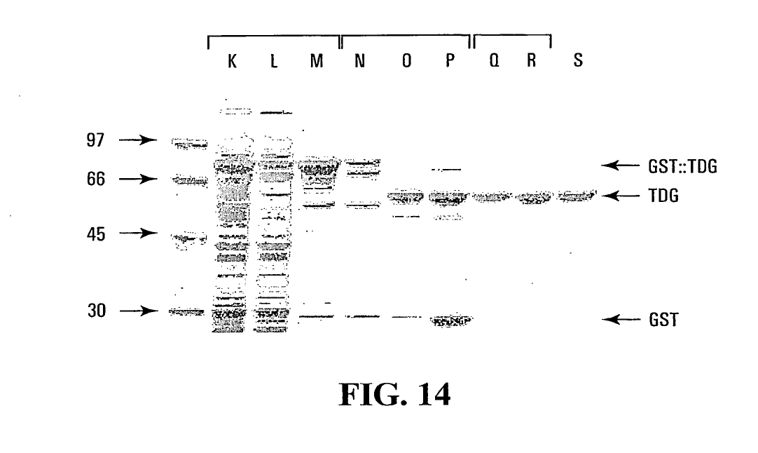

FIG. 14 represents SDS-PAGE analysis of the protein samples from mouse TDG purification K) Bacterial lysate, (L) Flow through HiPrep Heparin 16/10 column, (M) 60% B elution from HiPrep Heparin 16/10 column, (N) Flow through GSTPrep 16/10 column, (O) on-column cleaved product, (P) glutathione elution, (O) protein purified by salt gradient on Resource Q 6 ml column, (R) concentrated protein by 40% B step on Resource Q 6 ml column, (S) concentrated fractions from HiLoad 16/60 Superdex 200 prep colurmn.

The modification of the TDG protein can be post-translationally modified to confer a specific biological function. U. Hardeland, R. Steinacher, J. Jiricny and P. Schar. EMBO J. 2002 21(6) p.1456-64 (Modification of the human thymine-DNA glycosylase by ubiquitin-like proteins facilitates enzymatic turnover) describe the biologically relevant modification. The production of the specific modified TDG is important for selected applications, and the production of suitable amounts of the modified target is a difficult task. The method provided by the invention allows for the post-translational modification of endogenous and/or recombinant TDG and using either programmed lysate, reconstituted enzymatic components, and/or a combination of the two, can provide a sumoylation post-translational modifying event to the immobilized TDG molecule.

1. A method for post translational modification of a protein or polypeptide in the presence of a modifying composition capable of providing at least one modification comprising brining a liquid phase including the protein or polypeptide and a liquid extract of eukaryote or prokaryote cells into contact with a solid phase to immobilize the protein or polypeptide, and bringing the resultant solid phase carrying the immobilized protein or polypeptide at least once into contact with a liquid phase including the modifying composition to permit such modification to occur.

2. The method of claim 1, wherein the protein or polypeptide is a recombinant protein or polypeptide produced in a host cell.

3. The method of claim 1, wherein the extract is brought into contact with the solid phase without preliminary purification.

4. The method of claim 1, wherein the modifying composition includes at least one enzyme capable of catalyzing a modification reaction of the protein or polypeptide and any other components necessary for modifying the protein or polypeptide.

5. The method of claim 1, further comprising freeing the modified protein or polypeptide from the solid phase and collecting the freed protein or polypeptide by bringing the solid phase carrying the modified immobilized protein or polypeptide into contact with a liquid phase.

6. The method of claim 1, wherein the liquid phase including the composition capable of modifying the protein or polypeptide is brought into contact with the solid phase carrying the immobilized protein or polypeptide more than once.

7. The method of claim 6, wherein the liquid phase comprising the composition capable of modifying the protein or polypeptide is recirculated in contact with the solid phase carrying the immobilized protein or polypeptide.

8. The method of claim 1, wherein the protein or polypeptide is associated with a moiety capable of selective immobilization on the solid phase.

9. The method of claim 1, wherein the solid phase is an affinity resin.

10. The method of claim 9, wherein the protein or polypeptide is a recombinant protein fused to a protein or peptide fragment having affinity for the solid phase.

11. The method of claim 1, wherein the solid phase is an ion exchange resin

12. The method of claim 1, wherein the modification of the protein or polypeptide is selected from the group consisting of acylation, phosphorylation, dephosphorylation, SUMOylation, ubiquitinylation, carboxymethylation, formylation, acetylation, deacetylation, gamma carboxyglutamic acid, norleucine, amidation, deamidation, carboxylation, carboxyamylation, sulfation, methylation, demethylation, hydroxylation, ADP-ribosylation, maturation, adenylation, O-linked glycosylation, N-linked glycosylation, methonine oxidation, and addition of lipid (prenylation).

13. The method of claim 12, wherein the composition capable of modifying the protein or polypeptide includes at least one kinase.

14. The method of claim 12, wherein the protein or polypeptide includes at least one source of SUMO protein, wherein the composition capable of modifying the protein or polypeptide includes at least one enzyme capable of catalyzing SUMOylation.

15. The method of claim 12, wherein the protein or polypeptide has at least one lysine residue, in the presence of at least one source of ubiquitin, wherein the composition capable of modifying the protein or polypeptide includes at least one of the enzymes of the ubiquitination multi-enzyme system.

16. The method of claim 12, wherein the protein or polypeptide has at least one cysteine residue, and the composition capable of modifying the protein or polypeptide includes a carboxymethylase.

17. The method of claim 12, wherein the protein or polypeptide includes at least one residue selected from a lysine residue and an arginine residue, and the composition capable of modifying the protein or polypeptide comprises an acetyltransferase or a methyltransferase.

18. The method of claim 12, wherein the composition capable of modifying the protein or polypeptide includes an acyltransferase/amidase.

19. (cancelled)

20. The method of claim 12, wherein the protein or polypeptide includes at least one residue selected from the group consisting of an asparagine residue in the sequence Asn-X-Ser/Thr, a serine residue and a threonine residue and the composition capable of modifying the protein or polypeptide comprises a glycosylase.

21. A kit for modification of a protein or polypeptide, comprising components for immobilizing the protein or polypeptide on a solid phase and at least one component for modifying the immobilized protein.

22. The kit of claim 21, including components for freeing and collecting the modified protein or polypeptide from the solid phase.

23-25. (canceled)
